SEQUENCE LISTING (2 sequences)

SEQ ID NO: 1 - protein, 10 aa, Homo sapiens
SLLMCWITQC

SEQ ID NO: 2 - protein, 13 aa, homo sapiens
CAARSLLMWITQV

(19) United States
(12) Patent Application Publication
(10) Pub. No.: US 2010/0150952 A1
(43) Pub. Date: Jun. 17, 2010

(54) pH-RESPONSIVE POLYMER CARRIER COMPOSITIONS FOR CYTOSOLIC PROTEIN DELIVERY

(75) Inventors: Patrick S. Stayton, Seattle, WA (US); Suzanne Foster, Seattle, WA (US); Allan S. Hoffman, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UNIVERSITY OF WASHINGTON, Seattle, WA (US)

(21) Appl. No.: 12/615,166

(22) Filed: Nov. 9, 2009

Related U.S. Application Data
(60) Provisional application No. 61/112,594, filed on Nov. 7, 2008.

(51) Int. Cl. A61K 39/385; C08H 1/00; A61P 35/00

(52) U.S. Cl. 424/193.1; 526/238.1

(57) ABSTRACT

pH-Responsive polymer-based protein delivery carriers and compositions, methods for making the carriers and compositions, and methods for using the carriers and compositions for intracellular protein antigen delivery, inducing a cytotoxic T-lymphocyte response, introducing a tumor-specific protein antigen to an antigen presenting cell to induce an immune response, and providing tumor protection to a subject.

### CROSS-REFERENCE TO RELATED APPLICATION

This application claims the benefit of U.S. Provisional Application No. 61/112,594, filed Nov. 7, 2008, expressly incorporated herein by reference in its entirety.

### STATEMENT OF GOVERNMENT LICENSE RIGHTS

This invention was made with Government support under Contract No. RO1EB002991 awarded by the National Institutes of Health. The Government has certain rights in the invention.

### BACKGROUND OF THE INVENTION

There remains considerable opportunity for the improvement of cancer treatment strategies, and cancer vaccination, or immunotherapy, is a promising technique which employs the body's own immune system to identify and destroy diseased cells. This strategy provides the potential for increased efficacy over conventional methods, and if used in combination therapy could reduce the dose of toxic chemotherapeutic agents and radiation measures that are often accompanied by devastating side effects. Immunotherapy is especially promising in the prevention of the metastatic spreading of cancerous cells, which remains a significant threat even after the successful removal of primary tumor tissue by standard methods. Improving the efficacy and applicability of immunotherapy techniques could therefore lead to improved prognosis and quality of life for cancer patients undergoing treatment.

Suitable antigens have been identified for vaccination strategies, but effective delivery of these antigens to elicit a potent immune response remains a challenge. While substantial research has been performed in this area, an optimal carrier has yet to be developed. One considerable challenge is the need for efficient delivery of antigens into the Class I antigen presentation pathway, accessed primarily in the cytosol, of antigen presenting cells. This pathway leads to activation of cytotoxic T-lymphocytes (CTLs), which are capable of direct lysis of diseased cells and are critical to an effective immunotherapeutic response. The proposed research aims to increase the efficacy of therapeutic vaccination by developing a pH-sensitive carrier designed to increase the cytosolic delivery of antigens, enhancing the CTL immune response.

The traditional concept of a vaccine refers to immunization against bacterial or viral antigens prior to exposure to the actual pathogenic organism. Most conventional vaccines provide protection primarily through an antibody-mediated humoral immune response, and to some degree by stimulation of T-helper cells. The fundamentals of vaccine strategy have since been applied to cancer treatment. While a humoral immune response has been associated with a favorable prognosis in some cancers, research has mainly been focused on enhancing the cellular (T-cell-mediated) immune response. The activation of CTLs is a particular objective, as they are the immune component primarily responsible for eliminating tumor cells, as well as cells infected by some viruses. Unlike the prophylactic nature of traditional vaccines, cancer vaccination may be most valuable clinically as a therapeutic measure against previously established tumors. However, uses as a preventative technique may be applicable in cases of early identification of pre-cancerous lesions, for individuals with an increased genetic risk of certain cancers, or in preventing the metastatic spreading of a previously removed tumor.

Many tumors are characterized by the over-expression of tumor-specific protein antigens, and the goal of cancer vaccination is to identify these antigens and deliver them to the appropriate antigen presenting cells (APCs) in such a way as to induce an immune response against the antigen and any cancerous cells expressing it. Professional APCs possess the co-stimulatory molecules necessary to activate the T cells they interact with, resulting in an immune response against the antigen, rather than immune tolerance.

The professional antigen presenting cells of the immune system include dendritic cells, macrophages, and B cells. These cells process and present protein antigens either generated inside the cell (e.g. due to viral infection) or brought into the cell by sampling of the extracellular environment. B cells primarily internalize antigen that is bound to their IgG surface receptors. Macrophages are extraordinarily efficient at endocytosing antigens, including phagocytosis of large particulate substances. They present antigens on both MHC1 and MHC2 molecules, but are not as active at antigen presentation as dendritic cells. Dendritic cells (DCs) are derived from bone marrow and reside primarily in the peripheral tissues in their immature state, in which they are highly phagocytic. Dendritic cells are able to effectively internalize extracellular substances via phagocytosis (a receptor-dependent uptake of particulate substances), and macropinocytosis (the engulfing of surrounding extracellular fluid). DCs are stimulated to undergo maturation by inflammatory or pathogenic substances including bacterial lipopolysaccharides (LPS), heat shock proteins, and cytokines such as IL-1, GM-CSF, and TNFα; and such substances, which also stimulate macrophages, can be co-delivered as adjuvants in immunotherapy treatments. Mature DCs express a high number of MHC I and II molecules, as well as T-cell co-stimulatory molecules, and are able to migrate to secondary lymphoid organs such as the spleen and lymph nodes where they can interact with T-cells. Their ability to efficiently process and display antigens for T-cell recognition makes them a desirable in vivo target for cancer immunotherapy.

There are two main pathways by which antigens are processed and presented by APCs for recognition by T-cells. These pathways are depicted in FIG. 1. The class I pathway results in presentation of antigen fragments by major histocompatibility complex 1 (MHC1) molecules (in humans, the corresponding MHC molecules are the human leukocyte antigen, or HLA, molecules). MHC1 molecules interact with CD8⁺ receptors, leading to activation of cytotoxic T-lymphocytes (CTLs). CTLs are able to directly lyse diseased cells and play a critical role in effective immunotherapy. The MHC1 pathway is primarily entered via the cytoplasm of the APC. Proteins are degraded in the proteasome then further trimmed by cytosolic peptidases to generate MHC1 peptide epitopes. These peptides enter the endoplasmic reticulum (ER) via the TAP (transporter associated with antigen processing) transporter and combine with MHC 1 molecules. After folding of the MHC1-peptide complex is complete, it is transported to the cell surface and displayed to naïve T-cells.

The class II presentation pathway is the main pathway by which exogenous antigens are displayed. These antigens are presented on MHC2 molecules, which can bind to and activate CD4⁺ helper T-lymphocytes. Activated helper T-cells release cytokines that enhance the immune response and are important to maintaining CTL activity. The combination of MHC2-restricted peptide epitopes with MHC2 molecules occurs within the endocytic compartment of the cell.

Both macrophages, and, to a greater extent, DCs have been shown to exhibit some degree of cross-presentation, a mechanism by which extracellular antigens are presented by MHC1 molecules. The exact details of this process have yet to be determined, but several possible mechanisms are under investigation including: (1) a direct transport pathway from the endosome to the cytosol; (2) the delivery of a fraction of internalized antigen to the ER, where it can then be transported to the cytosol via TAP; (3) loading of a subset of MHC1 molecules within the endocytic pathway; and (4) the involvement of a portion of the ER membrane in the process of phagocytosis. Cross-presentation has been shown to be most efficient in the case of particulate substances taken up by the process of phagocytosis. While cross-presentation can occur in some instances, there remains a considerable opportunity for increasing the efficiency of antigen delivery to the cytosol in CTL-based immunotherapeutic strategies. While stimulation of both CD4⁺ and CD8⁺ T-cells is required to generate an effective immune response, activation of CTLs leads to potent antitumor activity and presents a greater challenge to immunotherapy.

Antigens can be delivered as whole proteins, peptide antigenic epitopes, or mRNA or DNA encoding a protein antigen. Delivery of DNA/mRNA provides the opportunity for an increased supply of antigen since it is translated multiple times upon reaching the nucleus/cytoplasm. Some disadvantages to these systems include the added requirement of DNA delivery into the nucleus, and the high susceptibility of mRNA to nuclease degradation, as well as the challenge its charge presents to delivery carrier formulation. The present research focuses on developing techniques for delivering both proteins and peptides, as the polymer carrier can be adapted to accommodate both. Delivery of full proteins is desirable because they are broken down in the proteasome to produce a range of possible MHC1 and MHC2 antigenic epitopes. This is useful because, due to HLA genetic variability, not all individuals will possess an equivalent set of HLA molecules. Delivery of whole protein antigens also allows the opportunity to activate a greater number of antigen-specific T-cells. However, obtaining full protein antigens in large enough amounts for therapeutic treatments can be expensive and time consuming. Much work has been done in determining the actual peptide epitopes which are displayed by the MHC molecules. The main limitation of this strategy is that it does not include all the variations of the antigen, limiting its usefulness across all members of the population and decreasing its chances of immune recognition. However, delivery of peptides is attractive because they can be synthesized chemically, making them considerably more accessible than full proteins. Furthermore, the sequences can be enhanced so that they better fit into the MHC complex, leading to increased T-cell activation.

Several approaches have been taken toward increasing antigen delivery in immunotherapy procedures, but there is much potential for improvement. One method involves removing dendritic cells from the patient and pulsing them with antigen, then re-injecting them. However, disadvantages include its high cost and the logistical complications stemming from the isolation and ex-vivo manipulation of the dendritic cells. Direct in vivo injection of antigen is advantageous due to its reduced complexity, lower cost, and decreased invasiveness. Since delivery of antigen alone has proven largely insufficient in inducing an immune response, a range of carriers are under development to enhance the delivery and uptake of injected antigens. Recombinant viral vectors, which take advantage of the efficient cellular transfection mechanisms of viruses, have been employed to deliver a variety of antigens, and conjugation to various bacterial toxins have been employed for similar reasons. Lipid-based carriers have also been explored for vaccine delivery. However, certain undesirable qualities of these techniques, such as safety concerns associated with viral vectors and toxicity and instability often observed with liposomal systems, has led to the exploration of synthetic polymer carriers.

Particles formed from poly(D,L-lactic-co-glycolic acid) (PLGA) have been widely investigated for antigen delivery and have resulted in increased MHC1 presentation of encapsulated antigens, a result of more efficient cross-presentation observed for particulate substances taken up by phagocytosis compared to soluble substances, as well as the adjuvant effect observed for PLGA particles. While some endosomal escape has been observed for this system, a mechanism for cytosolic delivery is not well established. PLG particles modified to have a cationic surface have also been explored for the delivery of DNA vaccines. Additionally, nanoparticulate carriers based on poly(y-glutamic acid)-poly(L-phenylalanine ethyl ester) have also been developed, and small 25 nm polypropylene sulfide nanoparticles have been formulated to specifically target dendritic cells in lymph nodes. One approach that has been employed to enhance the CTL response to delivered vaccines is the incorporation of MHC1 adjuvants, such as CpGDNA or TLR ligands, into carrier formulations. A few groups have investigated an approach similar to the one presented in this thesis, utilizing pH-responsive carriers to enhance the CTL response. One such study uses microparticles composed of dipalmitoylphosphatidylcholine and polymethacrylate Eudragit E100 to deliver antigenic peptides, and another system utilizes acid degradable particles and microgels for protein antigen delivery.

A significant opportunity for improving vaccine antigen delivery exists in the enhancement of endosomal escape, especially in the case of soluble, non-particulate antigen complexes. Exogenous substances entering cells by endocytosis are usually degraded, sequestered, or exocytosed in the endosomal-lysosomal pathway without gaining access to the cytoplasm. Enhancing the escape of therapeutic protein antigens from endosomal compartments could significantly increase the efficacy of protein vaccines through increased activation of CTLs. This approach is depicted schematically in FIG. 2.

Several groups have explored the use of pH-sensitive polymer-based delivery systems that take advantage of the decreased pH in the endosome. A popular approach to induce pH-responsive endosomal release has been the use of systems based on cationic polymers, which enhance endosomal release via the “proton sponge effect.” According to this mechanism, the polymers become protonated at endosomal pH which leads to an increased flux of ions into the endosome, causing an increase is osmotic pressure and eventual disruption of the endosome. However, the high toxicity associated with the delivery of polycationic substances presents a considerable challenge to these systems.

In an effort to enhance endosomal escape and reduce cytotoxicity, a variety of other polymers with membrane-active capabilities have been investigated. Carrier particles containing acid-degradable linkages, such as acetal, ketal, or hydrazone bonds have been developed to degrade more rapidly in the acidic endosomal environment to trigger release of their cargo. Several of these systems are being explored for use in antigen delivery. Block copolymers based on PEG-poly[(N″-citraconyl-2-aminoethyl)aspartamide] have been used to form nanocarriers which degrade at endosomal pH and release their cargo due to repulsive electrostatic force. Block ionomer complexes composed of graft-comb copolymers of Pluronic (PEO-PPO-PEO) and poly(acrylic acid) (Pluronic-PolyAA), and a model cationic surfactant, hexadecyltrimethylammonium bromide, have demonstrated an increase in positive charge at endosomal pH, which could potentially result in endosomal membrane interaction. Additionally, polymeric micelles based on poly(L-lactic acid), PEG, and poly(L-Histidine) and on PEG-poly(L-cystine bisamide-g-sulfadiazine) have been developed to target the lowered pH environment associated with tumors for the delivery of hydrophobic anticancer drugs.

Despite the advances noted above, a need exists for protein antigen delivery compositions and methods. The present invention seeks to fulfill this need and provides further related advantages.

### SUMMARY OF THE INVENTION

The present invention provides protein delivery carriers and compositions, methods for making the carriers and compositions, and methods for using the carriers and compositions for intracellular protein delivery.

In one aspect, the invention provides a polymer conjugate, comprising a pH-responsive polymer and one or more therapeutic agents covalently coupled thereto.

The pH-responsive polymer is a membrane destabilizing polymer or membrane disrupting polymer. In one embodiment, the pH-responsive polymer comprises a plurality of repeating units comprising a C2-C8 alkyl group and a carboxylic acid group ionized at pH 7.4 and protonated at pH 5.5-6.0.

The therapeutic agent is a protein or peptide therapeutic agent. In one embodiment, the therapeutic agent is an immunotherapeutic agent, such as a protein or peptide antigen. In one embodiment, the immunotherapeutic agent is a protein or peptide cancer antigen. In one embodiment, the immunotherapeutic agent is a protein or peptide human cancer antigen. In one embodiment, the immunotherapeutic agent is a protein or peptide vaccine.

In another aspect, the invention provides a particle comprising the conjugate of the invention and a cationic complexing agent.

In a further aspect, the invention provides pharmaceutical compositions. The pharmaceutical composition comprises a pharmaceutically acceptable carrier and a conjugate or particle of the invention.

In another aspect, the invention provides a method for delivering a protein or peptide antigen to cell's cytosol, comprising contacting a cell with a conjugate or a particle of the invention.

In another aspect, the invention provides a method for inducing a cytotoxic T-lymphocyte response, comprising contacting a cell with a conjugate or a particle of the invention.

In another aspect, the invention provides a method for providing tumor protection to a subject, comprising administering to a subject a therapeutically effective amount of the conjugate of a conjugate or a particle of the invention, wherein the immunotherapeutic agent is a protein or peptide cancer antigen.

In another aspect, the invention provides a method for introducing a tumor-specific protein antigen to an antigen presenting cell to induce an immune response against the antigen and cells presenting the antigen, comprising contacting an antigen presenting cell with a conjugate or a particle of the invention.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1. Summary of MHC-1 and MHC2 pathways. The MHC-1 pathway leads to activation of CD⁸+ cytotoxic T-lymphocytes and typically presents antigens that are processed in the cytoplasm. The MHC2 pathway leads to activation of CD⁴+ helper T-lymphocytes and typically presents exogenous antigens that are internalized into endosomes.

FIG. 2. Schematic representation of possible endosomal escape mechanism provided by pH-responsive smart polymers to deliver biotherapeutics (purple) to the cytosol.

FIG. 3. Structure of the PPAA-PDSA polymer. Poly(propyl acrylic acid) (PPAA) is the primary carrier component and becomes increasingly protonated as the pH decreases, converting from anionic and hydrophilic at physiological pH to protonated and hydrophobic at the lower pH observed in the endosome. This enables PPAA to become membrane interactive at endosomal pH. Pyridyldisulfide acrylate (PDSA), is incorporated into the polymer at 3-5 mol %, and provides a conjugation site via disulfide exchange reaction.

FIG. 4. Schematic of PPAA-PDSA-Ovalbumin conjugation chemistry for conjugation of ovalbumin to the PDSA moiety of the PPAA-PDSA polymer. The protein is first modified using Traut's reagent to convert primary amines to thiols, then the polymer is added and a disulfide exchange reaction occurs, releasing pyridine-2-thione. Reaction progress can be monitored by measuring A₃₄₃ of released pyridine-2-thione.

FIG. 5. Glutathione reversibility of the polymer-protein conjugation. In the absence of the cytosolic reducing agent glutathione (A), little free ovalbumin is present. However, in the presence of 10 mM glutathione (B), the ovalbumin band reappears due to reduction of the disulfide bond attaching it to the polymer.

FIG. 6. pH-dependent hemolysis properties of PPAA polymers and ovalbumin conjugates. Red blood cells were isolated and added to polymer and conjugate solutions (polymer concentrations=5 μg/ml) in 0.1M phosphate buffer. Hemolysis is reported as a percentage of complete lysis by Triton X-100. PPAA-PDSA shows high membrane-disruptive activity at pH 5.8 but considerably lower membrane disruption at pH 7.4. The polymer retains its hemolysis capabilities after conjugation to the hydrophilic protein ovalbumin. PMAA-PDSA, however, does not lyse red blood cells at any pH due to the decreased hydrophobicity of its methyl side chain compared with the propyl side chain of PPAA.

FIG. 7A. CTL activation/MHC-1 presentation of PPAA-ovalbumin conjugates. Samples were incubated with RAW macrophages for 6 hrs then removed and B3Z T-cells were added for 16 hrs. Cells were rinsed and incubated 4 hrs with lysis buffer containing chlorophenol red β-D-galactoside, then absorbance of released chlorophenol red was measured at 595 nm. Samples were evaluated in triplicate and errors are reported as +/−one standard deviation. For reference, the maximum possible β-galactosidase production was determined by chemically stimulating the B3Z cells using PMA/ionomycin for 4 hrs, which gave an A₅₉₅ of 0.56. Ovalbumin concentration=100 μg/ml. The PPAA-ovalbumin conjugate shows significantly greater CTL activation than do any of the control samples (p<0.0005). This is likely due to the endosomal disruption provided by PPAA, which allows the protein to more efficiently access the MHC-1 pathway in the cytoplasm. However, PMAA conjugation results in low CTL activation, similar to that for free ovalbumin or a PPAA ovalbumin physical mixture (p=0.2). This suggests that the increase in MHC-1 presentation provided by PPAA is due to its endosomal disruptive properties rather than solely to increased uptake due to its larger size compared to free ovalbumin.

FIG. 7B. Dose-Dependent CTL activation induced by PPAA-ovalbumin conjugates. Samples were incubated with RAW macrophages at 3 concentrations prior to evaluation with the B3Z CT1s. Samples were evaluated in triplicate and errors are reported as +/−one standard deviation. The PPAA-ovalbumin conjugates enhance CTL stimulation in a dose-dependent manner. It can also be seen that when the polymer ratio is increased from 1.7 μg/μg ova to 3.2 μg/μg ova, maximal CTL activation is reached at a lower ovalbumin concentration.

FIG. 8. Cytotoxicity of PPAA and PMAA polymers and conjugates. Cytotoxicity was determined for both RAW and B3Z cells using the LDH assay. Samples were added to cells for 24 hrs at a concentration of 300 μg/ml polymer, twice the highest concentration used in the MHC-1 presentation assay. The cell supernatant was then combined with LDH reagent and the absorbance at 490 nm was recorded. Percent survival=1−[(A₄₉₀ of sample−A₄₉₀ of untreated cells control)/(A₄₉₀ of TritonX control−A₄₉₀ of untreated cells control)]×100%. Samples were evaluated in triplicate and error is expressed as +/−SEM. It can be seen that excessive toxicity was not observed for any of the samples.

FIG. 9. Uptake of ¹⁴C-Ovalbumin-PPAA by macrophages increases over time. RAW macrophages were incubated with samples at a concentration of 50 μg/ml ovalbumin. Cells were washed with PBS and lysed using 1% Triton X-100. Radioactivity in the cell media, PBS wash, and cell lysate was measured, and uptake of ¹⁴C-ovalbumin was calculated as the % radioactivity present in the cell lysate compared to the total radioactivity delivered. Experiments were performed in triplicate and error is expressed as +/−one standard deviation. It can be seen that PPAA-conjugated ovalbumin continually accumulates in the cell, whereas the control sample levels remain fairly constant (p>0.09). This is likely due to the ability of PPAA-ovalbumin to escape the endosome before being exocytosed.

FIG. 10A. Exocytosis study: Total amount of ¹⁴C-ovalbumin internalized during the 1-min uptake time. Samples were incubated with RAW macrophages for 1 min, then un-internalized conjugate was washed off. The exocytosis of ¹⁴C-ovalbumin into fresh supernatant was followed for 4 hrs, followed by cell lysis. The amount of ovalbumin internalized after a 1-min incubation time was determined from the combined exocytosed and cell-associated radioactivity, as a percentage of the total delivered. Samples were evaluated with a minimum of n=3 and error is reported as +/−SEM. It can be seen that the amount of ovalbumin taken into the cells in 1 min is similar for all samples, giving a uniform starting point for the exocytosis measurements.

FIG. 10B. Exocytosis study: Exocytosis profiles following 1-min uptake time. Following the 1-min incubation of samples with RAW macrophages, un-internalized conjugate was washed off and the reappearance of ¹⁴C-ovalbumin into fresh supernatant was measured at various time intervals. After 4 hrs, cells were lysed with 1% Triton X-100 and the radioactivity in the lysate was measured. The amount of ovalbumin exocytosed at each timepoint was calculated as a percentage of the amount initially internalized. Samples were evaluated with a minimum of n=3 and error is reported as +/−SEM. It can be seen that most exocytosis occurred in the first 30 minutes, and exocytosis rates were similar for all samples. However, much less PPAA-conjugated ovalbumin was exocytosed at each timepoint, compared to controls.

FIG. 10C. Exocytosis study: Amount of initially internalized ¹⁴C-ovalbumin remaining in the cells after 4 hrs exocytosis. Following the 1-min incubation of samples with RAW macrophages, un-internalized conjugate was washed off and the reappearance of ¹⁴C-ovalbumin into fresh supernatant was measured at various time intervals. After 4 hrs, cells were lysed with 1% Triton X-100 and the radioactivity in the lysate was measured. The amount of ¹⁴C-ovalbumin remaining in the cells after 4 hrs of exocytosis was calculated as a percentage of the amount initially internalized. Samples were evaluated with a minimum of n=3 and error is reported as +/−SEM. The amount of ovalbumin that remained in the cells and was not exocytosed was greatly enhanced for PPAA conjugates. This effect is likely due to the ability of the polymer to disrupt the endosomal membrane and deliver the ovalbumin to the cytosol before exocytosis can occur.

FIG. 11A. Exocytosis study: Total amount of ¹⁴C-ovalbumin internalized during the 15-min uptake time. This experiment is similar to that shown in FIG. 9 except samples were incubated with RAW macrophages for 15 min rather than 1 min. After 15 min, un-internalized conjugate was removed and the exocytosis of ¹⁴C-ovalbumin into fresh supernatant was followed for 4 hrs, followed by cell lysis. The amount of ovalbumin internalized after the 15-min incubation time was determined from the combined exocytosed and cell-associated radioactivity, as a percentage of the total delivered. Samples were evaluated with a minimum of n=3 and error is reported as +/−one standard deviation. The preferential accumulation of PPAA-ovalbumin is observed for this longer incubation time.

FIG. 11B. Exocytosis study: Exocytosis profiles following a 15-min uptake time. Following the 1-min incubation of samples with RAW macrophages, un-internalized conjugate was washed off and the reappearance of ¹⁴C-ovalbumin into fresh supernatant was measured at various time intervals. After 4 hrs, cells were lysed with 1% Triton X-100 and the radioactivity in the lysate was measured. The amount of ovalbumin exocytosed at each timepoint was calculated as a percentage of the amount initially internalized. Samples were evaluated with a minimum of n=3 and error is reported as +/−one standard deviation. Although the initial amounts were different for each sample at the beginning of the study, after normalization to these initial amounts it can be seen that the exocytosis profiles are very similar to those shown in FIG. 9 for the 1-min uptake time.

FIG. 11C. Exocytosis study: Amount of initially internalized ¹⁴C-ovalbumin remaining in the cells after 4 hrs exocytosis. Following the 15-min incubation of samples with RAW macrophages, un-internalized conjugate was washed off and the reappearance of ¹⁴C-ovalbumin into fresh supernatant was measured at various time intervals. After 4 hrs, cells were lysed with 1% Triton X-100 and the radioactivity in the lysate was measured. The amount of ¹⁴C-ovalbumin remaining in the cells after 4 hrs of exocytosis was calculated as a percentage of the amount initially internalized. Samples were evaluated with a minimum of n=3 and error is reported as +/−one standard deviation. The percentage of ovalbumin remaining in the cells was again greatly enhanced for PPAA conjugates after this longer initial incubation.

FIG. 12. Schematic of PPAA-Ova/pDMAEMA ionic particle formation. The structure of the cationic complexing agent DMAEMA is shown in the top panel. The 10 kD pDMAEMA used in this study is about 50% protonated at pH 7.4. The lower panel shows a schematic of the particle formulation process.

FIG. 13. No cytotoxicity is observed for PPAA and PMAA conjugates or particles. Toxicity was studied in RAW macrophages using the Alamar blue assay. Samples were incubated for 24 hrs at a concentration of 750 μg/ml polymer, 5× the amount used for the therapeutic studies. % survival is shown as a percentage of the untreated cells.

FIG. 14. Uptake of ¹⁴C-Ova is greater for PPAA particles than for the soluble conjugate. RAW macrophages were incubated with samples at a concentration of 50 μg/ml of ovalbumin. The cells were then washed with PBS and lysed using 1% Triton X-100. Radioactivity in the cell media, PBS wash, and cell lysate was measured, and uptake of ¹⁴C-ovalbumin was calculated as the % radioactivity present in the cell lysate compared to the total radioactivity delivered. Experiments were performed in triplicate and error is expressed as +/−one standard deviation. It can be seen that PPAA-ova particle accumulates with time, to a greater extent than both the soluble PPAA conjugate and the control PMAA particle.

FIG. 15A. Exocytosis Study: Exocytosis profiles of ¹⁴C-ovalbumin conjugates and particles after a 15 min uptake time. Samples were incubated with RAW macrophages for 15 min, then un-internalized samples were removed and cells were washed 2× with media. Fresh media was added at various time intervals and the reappearance of ¹⁴C-ovalbumin into the supernatant was measured. After 4 hrs, cells were lysed with 1% Triton X-100 and the radioactivity in the lysate was measured. The amount of ovalbumin exocytosed at each timepoint was calculated as a percentage of the amount initially internalized. Samples were evaluated with a minimum of n=3 and error is reported as +/−one standard deviation. This figure and the following show the increased intracellular accumulation/decreased exocytosis achieved by PPAA conjugates, and to a greater extent PPAA particles. PMAA particles also show some retention compared to the PMAA conjugate.

FIG. 15B. Exocytosis study: Amount of initially internalized ¹⁴C-ovalbumin remaining in the cells after 4 hrs exocytosis. Following the 15-min incubation of samples with RAW macrophages, un-internalized conjugate was washed off and the reappearance of ¹⁴C-ovalbumin into fresh supernatant was measured at various time intervals. After 4 hrs, cells were lysed with 1% Triton X-100 and the radioactivity in the lysate was measured. The amount of ¹⁴C-ovalbumin remaining in the cells after 4 hrs of exocytosis was calculated as a percentage of the amount initially internalized. Samples were evaluated with a minimum of n=3 and error is reported as +/−one standard deviation. This figure summarizes the increased intracellular accumulation/decreased exocytosis achieved by PPAA conjugates and to a greater extent PPAA particles, as well as the intermediate retention achieved by the PMAA particles.

FIG. 16. Amount of internalized ¹⁴C-ovalbumin after a 15 min uptake time. The amount of ovalbumin internalized after a 15 min incubation time was determined as a percentage of the total delivered. Samples were evaluated with a minimum of n=3 and error is reported as +/−one standard deviation. The preferential accumulation of PPAA-ovalbumin is observed for this longer incubation time.

FIG. 17. CTL activation/MHC-1 presentation of PPAA-ovalbumin particles. RAW macrophages were incubated with samples at a 100 μg/ml ovalbumin concentration for 4 hrs then washed, and B3Z T-cells were added for 16 hrs. Cells were again washed and incubated for 2 hrs with lysis buffer containing chlorophenol red 13-D-galactoside, then the absorbance of released chlorophenol red was measured at 595 nm. The background signal of the untreated cells has been subtracted. Samples were evaluated in triplicate and errors are reported as +/−one standard deviation. It can be seen that the PPAA particles result in greatly enhanced CTL activation compared to the soluble PPAA conjugate and the free ovalbumin. A high concentration ovalbumin sample (5 mg/ml) was used as a positive control and gave an A₅₉₅ value of 0.46.

FIG. 18. Tumor growth curves for ovalbumin-vaccinated mice. C57B1/6 mice (4 per group) were vaccinated with PBS, ovalbumin, or soluble or particulate PPAA-ova (100 μg ova per mouse), and 7 days later were injected subcutaneously with E.G7-OVA tumor cells. Tumor size was measured until tumor volume exceeded 2000 mm³. Error bars represent the SEM. The excellent tumor protection provided by the PPAA carrier for the 4-week duration of the study is evidenced in this plot.

FIG. 19. Anti-ovalbumin IgG antibody concentration in mouse plasma. C57B1/6 mice (4 per group) were vaccinated with PBS, ovalbumin, or soluble or particulate PPAA-ova (100 μg ova per mouse). 20 days after vaccine injection, blood was drawn and ELISA was performed plasma samples. Error bars represent the SEM. The anti-ova antibody response induced by the PPAA soluble and particulate conjugates is much greater than that resulting from vaccination with free ovalbumin.

FIG. 20. Ovalbumin-reactive CD8+ splenocytes. C57B1/6 mice (4 per group) were vaccinated with PBS, ovalbumin, and soluble and particulate PPAA-ova (100 μg ova per mouse. 7 days later spleens were collected and splenocytes were stained with FITC-anti-CD8 and PE-MHC-1/SIINFEKL tetramers. Errors bars represent the SEM. The enhanced anti-ova CTL response induced by the PPAA soluble and particulate conjugate can be seen.

FIG. 21. Dose-Dependent CTL Activation/Class I antigen presentation by DCs pulsed with SLLMWITQC peptides. Primary DCs were incubated for 4 hours at increasing peptide concentrations. Maturation was then induced and CTLs were added for another 18 hours. IFN-γ production by activated CTLs was measured by ELISA. Samples were evaluated in duplicate. The PPAA-peptide provides dose-dependent CTL activation, though not to as great an extent as the free peptide.

FIG. 22. CTL Activation/Class I antigen presentation by DCs pulsed with CAARSLLMWITQV peptides conjugated to PPAA. Primary DCs were incubated with samples for 4 hours, then maturation was induced and CTLs were added for another 18 hours. IFN-γ production by activated CTLs was measured by ELISA. Samples were evaluated in duplicate, and the concentrations presented are those of the peptide. The modified peptide conjugated to PPAA results in some dose-dependent CTL activation, though not to as great an extent as the native unmodified peptide.

FIG. 23. CTL Activation/Class I antigen presentation by DCs using BFA inhibitor. Primary DCs were incubated with samples for 4 hours. Maturation was then induced and CTLs were added for another 18 hours. IFN-γ production by activated CTLs was measured by ELISA. Peptide concentration are 1 ug/ml for all samples except the native SLLMWITQV peptide, which is 0.1 ug/ml. Samples were evaluated in duplicate. Again, PPAA-peptide does not result in as much CTL activation as does the free peptide. Some reduction is seen when the BFA inhibitor is employed, but it is not substantial and occurs to the same extent for the free peptide samples as for the conjugate.

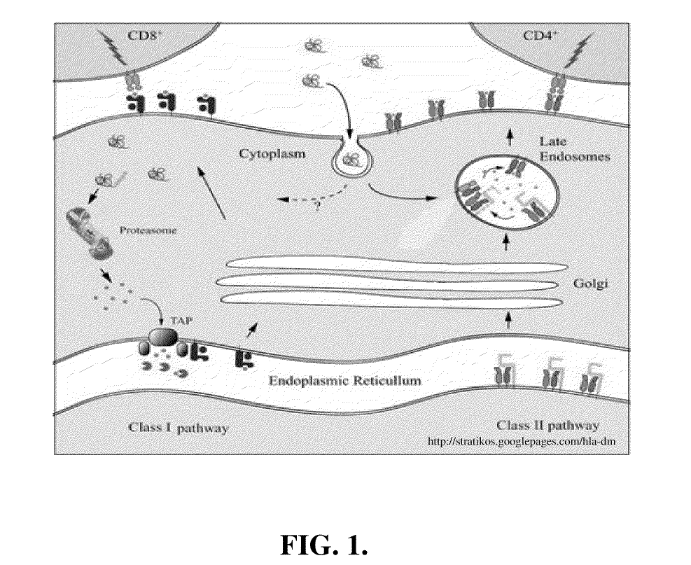

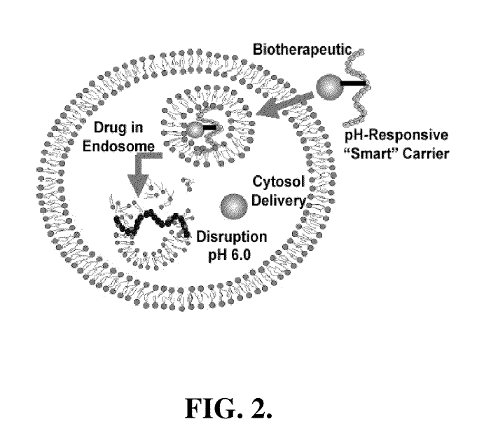

### DETAILED DESCRIPTION OF THE INVENTION

The present invention provides protein delivery carriers and compositions, methods for making the carriers and compositions, and methods for using the carriers and compositions for intracellular protein delivery.

In one aspect, the invention provides a polymer conjugate, comprising a pH-responsive polymer and one or more therapeutic agents covalently coupled thereto.

The pH-responsive polymer is a membrane destabilizing polymer or membrane disrupting polymer. In one embodiment, the pH-responsive polymer comprises a plurality of repeating units comprising a C2-C8 alkyl group and a carboxylic acid group ionized at pH 7.4 and protonated at pH 5.5-6.0. In one embodiment, the pH-responsive polymer comprises a repeating unit selected from C2-C8 alkyl acrylic acid repeating units. In one embodiment, the pH-responsive polymer comprises a repeating unit selected from ethyl acrylic acid and propylacrylic acid repeating units. In one embodiment, the pH-responsive polymer is a poly(propylacrylic acid). The pH-responsive polymer can be a random, block, or graft copolymer.

As used herein, the term “membrane destabilizing” refers to a polymer or composition that directly or indirectly elicit a change (e.g., a permeability change) in a cellular membrane structure (e.g., an endosomal membrane) so as to permit an agent (e.g., protein or peptide) to pass through such membrane structure, for example, to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or otherwise disrupt a cellular membrane (e.g., as observed for a substantial fraction of a population of cellular membranes). Generally, membrane destabilizing or membrane disruptive properties of polymers can be assessed by various means. In one non-limiting approach, a change in a cellular membrane structure can be observed by assessment in assays that measure (directly or indirectly) release of an agent (e.g., protein or peptide) from cellular membranes (e.g., endosomal membranes), for example, by determining the presence or absence of such agent, or an activity of such agent, in an environment external to such membrane. Another non-limiting approach involves measuring red blood cell lysis (hemolysis), e.g., as a surrogate assay for a cellular membrane of interest. Such assays may be done at a single pH value or over a range of pH values.

The terms “endosome disruptive” and “endosomolytic” refers to a polymer or composition having the effect of increase the permeability of the endosomal membrane of an endosome.

The phrase “pH-responsive, membrane-destabilizing or “pH-dependent, membrane-destabilizing” refers to a polymer or composition that is at least partially, predominantly, or substantially hydrophobic and is membrane destabilizing in a pH dependent manner. In certain instances, a pH-dependent membrane destabilizing polymer block is a hydrophobic polymeric segment of a copolymer and/or comprises a plurality of hydrophobic species; and comprises a plurality of chargeable species. In some embodiments, the chargeable species is anionic. In some embodiments, the anionic chargeable species is anionic at about neutral pH. In further or alternative embodiments, the anionic chargeable species is non-charged at a lower, e.g., endosomal pH. In some embodiments, the membrane destabilizing chargeable hydrophobe comprises a plurality of cationic species. The pH dependent membrane-destabilizing chargeable hydrophobe comprises a non-peptidic and non-lipidic polymer backbone. For example, a pH dependent, membrane-destabilizing block may possess anionic repeat units the substituents of which are predominantly ionized (anions) at one pH, e.g., pH 7.4, and predominantly neutral at a lesser pH, e.g., pH 5.0 whereby the pH dependent, membrane-destabilizing group or block becomes increasingly hydrophobic as a function of the drop in pH from 7.4 to 5.0.

The polymeric carrier described herein is a pH-dependent, membrane destabilizing carrier. The polymeric carrier can be a random, block, or graft copolymer.

The polymeric carrier is or comprises a plurality of monomeric anionic residues and, optionally one or more cationic, zwitterion, or neutral monomeric residues. In specific embodiments, the plurality of anionic monomeric residues is anionic at about neutral pH (e.g., physiological pH). In more specific embodiments, at least 50%, at least 70%, at least 80%, at least 90%, at least 95%, or at least 99% of the plurality of anionic monomeric residues are non-charged at about pH 5, at about pH 5.5, at about pH 5.7, at about pH 6.0, at about pH 6.2, at about pH 6.5, or at about endosomal pH (e.g., as calculated from the pKa value of the given monomeric residue).

In certain embodiments, the plurality of monomeric residues can be derived from polymerization of (C₂-C₈) alkyl ethacrylate, a (C₂-C₈) alkyl methacrylate, or a (C₂-C₈) alkyl acrylate (each of which may be optionally substituted).

The polymeric carrier may contain anionic repeat units, cationic repeat units, zwitterionic repeat units, a combination of two or more charged repeat units (e.g., anionic and cationic repeat units, anionic and zwitterionic repeat units, cationic and zwitterionic repeat units, or anionic, cationic and zwitterionic repeat units), substantially non-charged repeat units, or a combination thereof, provided that its overall character is pH-responsive and membrane disruptive. The polymeric carrier may contain any of a wide range of repeat units, hydrophobic or even hydrophilic, provided that the sum of the contributions of the repeat units comprised by the carrier provides a polymer having an overall pH-responsive character. When the repeat units contain ionizable groups, the contribution of an individual repeat unit to the overall hydrophilicity of the block of which it is a constituent may vary as a function of its pKa relative to the pH of the environment in which it is found. For example, propyl acrylic acid repeat units, —CH₂C(CH₂CH₂CH₃)(COOH)—, are predominantly ionized at pH 7 but not at pH 5 and thus, the hydrophobic contribution of propyl acrylic acid repeat units to a block is significantly greater at pH 5 than at pH 7. In general, therefore, it is preferred that the sum of the contributions of the repeat units constituting the polymeric carrier be such that the overall character of the block is hydrophobic at pH's that are less than physiological pH. For example, in one embodiment, the sum of the contributions is such that the overall character of the carrier is hydrophobic at a pH of about 5.0. By way of further example, in one embodiment, the sum of the contributions is such that the overall character of the carrier is hydrophobic at a pH of about 5.5. By way of further example, in one embodiment, the sum of the contributions is such that the overall character of the carrier is hydrophobic at a pH of about 6.0. By way of further example, in one embodiment, the sum of the contributions is such that the overall character of the carrier is hydrophobic at a pH of about 6.8. By way of further example, in one embodiment, the sum of the contributions of the repeat units is such that the overall character of the carrier is hydrophobic at a pH within the range of about 6.2 to 6.8.

In certain embodiments, the polymeric carrier described herein comprises monomeric residues resulting from the polymerization or copolymerization of a monomer comprising a hydrophobic species. Monomers comprising a hydrophobic species include, by way of non-limiting example, optionally substituted, (C₂-C₈)alkyl-ethacrylate, a (C₂-C₈)alkyl-methacrylate, a (C₂-C₈)alkyl-acrylate, styrene, (C₂-C₈)alkyl-vinyl, or the like.

In certain embodiments, the polymeric carrier described herein comprises a plurality of first monomeric residues derived from a first polymerizable monomer having a protonatable anionic species and a hydrophobic species, and optionally a plurality of second monomeric residues derived from a second polymerizable monomer having a deprotonatable cation species.

In one embodiment, the polymeric carrier comprises repeat units corresponding to formula (I):

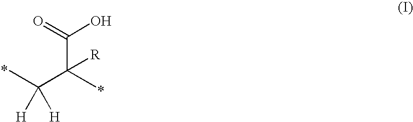

wherein * designates the point of attachment of the repeat unit of formula (I) to other repeat units; and R is a C2-C8 alkyl group. Representative C2-C8 alkyl groups include methyl, ethyl, and straight chain and branched propyl, butyl, pentyl, hexyl, heptyl, and octyl groups. In one embodiment, R is ethyl. In another embodiment, R is n-propyl.

In one embodiment, the polymeric carrier is a random copolymer comprising repeat units corresponding to formula (I). In general, the polymeric carrier comprises a plurality of repeat units, i.e., at least two. In certain embodiments, a the polymeric carrier described herein has a number average molecular weight of about 1,000 Dalton to about 200,000 Dalton, about 1,000 Dalton to about 100,000 Dalton, about 1,000 Dalton to about 100,000 Dalton, about 5,000 Dalton to about 50,000 Dalton, about 10,000 Dalton to about 50,000 Dalton, about 15,000 Dalton to about 35,000 Dalton, or about 20,000 Dalton to about 30,000 Dalton.

In another embodiment, the polymeric carrier is a block copolymer comprising ethylenically unsaturated monomers. The term “ethylenically unsaturated monomer” is defined herein as a compound having at least one carbon double or triple bond. Suitable ethylenically unsaturated monomers include alkyl (alkyl)acrylates, methacrylates, acrylates, alkylacrylamides, methacrylamides, and acrylamides.

In various embodiments, any monomer suitable for providing the polymer carriers can be used. In some embodiments, monomers suitable for use in the preparation of the polymers include, for example, one or more of the following monomers: methyl methacrylate, ethyl acrylate, propyl methacrylate (all isomers), butyl methacrylate (all isomers), 2-ethylhexyl methacrylate, methacrylic acid, benzyl methacrylate, phenyl methacrylate, methyl acrylate, ethyl acrylate, propyl acrylate (all isomers), butyl acrylate (all isomers), 2-ethylhexyl acrylate, acrylic acid, benzyl acrylate, phenyl acrylate, acrylates selected from glycidyl methacrylate, 2-hydroxyethyl methacrylate, hydroxypropyl methacrylate (all isomers), hydroxybutyl methacrylate (all isomers), or combinations thereof.

In some embodiments, polymer carriers have a low polydispersity index (PDI) or differences in chain length. Polydispersity index (PDI) can be determined in any suitable manner, e.g., by dividing the weight average molecular weight of the polymer chains by their number average molecular weight. The number average molecule weight is sum of individual chain molecular weights divided by the number of chains. The weight average molecular weight is proportional to the square of the molecular weight divided by the number of molecules of that molecular weight. Because the weight average molecular weight is always greater than the number average molecular weight, polydispersity is always greater than or equal to one. As the numbers come closer and closer to being the same, i.e., as the polydispersity approaches a value of one, the polymer becomes closer to being monodisperse in which every chain has exactly the same number of constitutional units. Polydispersity values approaching one are achievable using radical living polymerization. Methods of determining polydispersity, such as, but not limited to, size exclusion chromatography, dynamic light scattering, matrix-assisted laser desorption/ionization chromatography and electrospray mass chromatography are well known in the art. In some embodiments, the polymer carriers have a polydispersity index (PDI) of less than 2.0, or less than 1.8, or less than 1.6, or less than 1.5, or less than 1.4, or less than 1.3, or less than 1.2.

The polymer carriers useful in the invention are membrane-destabilizing at a pH of about 6.5 or lower, preferably at a pH ranging from about 5.0 to about 6.5, or at a pH of about 6.2 or lower, preferably at a pH ranging from about 5.0 to about 6.2, or at a pH of about 6.0 or lower, preferably at a pH ranging from about 5.0 to about 6.0. For example, in one embodiment, the polymer is membrane-destabilizing at a pH of or less than about 6.2, of or less than about 6.5, of or less than about 6.8, of or less than about 7.0. In certain embodiments, membrane destabilization is of any cellular membrane such as, for example, an extracellular membrane, an intracellular membrane, a vesicle, an organelle, an endosome, a liposome, or a red blood cell. In some embodiments, polymeric carriers are membrane destabilizing (e.g., in an aqueous medium) at an endosomal pH.

In some embodiments, the polymeric carrier is hemolytic at pH of or less than about 6.2, of or less than about 6.5, of or less than about 6.8, of or less than about 7.0. In further or alternative embodiments, the polymeric carrier is substantially non-hemolytic at pH greater than about 7.0. In specific embodiments, the polymeric carrier is hemolytic at given concentration and a pH of about 6.2, and substantially non-hemolytic at the same concentration and at a pH greater than about 7.0. In certain embodiments, the hemolytic nature of the polymer is determined in any suitable manner, e.g., by use of any standard hemolysis assay, such as an in vitro hemolysis assay.

In certain embodiments, the polymeric carrier is endosome disruptive. In some embodiments, the polymeric carrier is endosome disruptive at pH of or less than about 6.2, of or less than about 6.5, of or less than about 6.8, of or less than about 7.0. The endosome disruptive nature of the polymeric carrier is determined in any suitable manner, e.g., by use of any standard hemolysis assay, such as an in vitro endosomolysis assay, or an in vivo non-human mammal endosomolysis assay.

The invention provides a polymer conjugate, comprising a pH-responsive polymer and one or more therapeutic agents covalently coupled thereto. As used herein, therapeutic agent therapeutic agent refers to an agent that, when administered to a subject, organ, tissue, or cell has a therapeutic effect and/or elicits a desired biological and/or pharmacological effect. In one embodiment, the therapeutic agent is an immunotherapeutic agent, more specifically, a protein or peptide therapeutic agent. In one embodiment, the immunotherapeutic agent is a protein or peptide antigen. In one embodiment, the immunotherapeutic agent is a protein or peptide cancer antigen. In one embodiment, the immunotherapeutic agent is a protein or peptide human cancer antigen. In one embodiment, the immunotherapeutic agent is a protein or peptide vaccine.

The immunotherapeutic agent is covalently coupled to the polymer. In one embodiment, the immunotherapeutic agent is covalently coupled to the polymer through a disulfide linkage.

In another aspect, the invention provides a particle comprising the conjugate of the invention and a cationic complexing agent. Suitable cationic complexing agents include any cationic agent capable of associating with a conjugate of the invention to provide a particle (e.g., ionic particle). Representative cationic complexing agents include cationic polymers (e.g., pDMAEMA). In contrast to the conjugates of the invention, which are soluble in aqueous media, the particles of the invention are non-soluble.

In a further aspect, the invention provides pharmaceutical compositions. The pharmaceutical composition comprises a pharmaceutically acceptable carrier and a conjugate or particle of the invention.

Formulations comprising the pH-responsive polymer compositions of the invention (i.e., conjugates and particles) can be pharmaceutical compositions. Such pharmaceutical compositions can comprise, for example, a composition of the invention and a pharmaceutically acceptable excipient.

In some embodiments, the pH-responsive polymer composition is administered to a patient in any suitable manner, e.g., with or without stabilizers, buffers, and the like, to form a pharmaceutical composition. In some embodiments, the pH-responsive polymer composition is formulated and used as tablets, capsules or elixirs for oral administration, suppositories for rectal administration, sterile solutions, suspensions or solutions for injectable administration, and any other suitable compositions.

In some embodiments, pharmaceutical compositions comprising the pH-responsive polymer composition are administered systemically. As used herein, “systemic administration” means in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. In some embodiments, the compositions are administered topically.

In some embodiments, the compositions are prepared for storage or administration and include a pharmaceutically effective amount of the pH-responsive polymer composition in a pharmaceutically acceptable carrier or diluent. Any acceptable carriers or diluents are optionally utilized herein. Specific carriers and diluents and are described, e.g., in Remington's Pharmaceutical Sciences, Mack Publishing Co., A. R. Gennaro Ed., 1985. As used herein, the term “pharmaceutically acceptable carrier” means a non-toxic, inert solid, semi-solid or liquid filler, diluent, encapsulating material or formulation auxiliary of any type. In some embodiments, the pharmaceutical compositions provided herein are administered to humans and/or to animals, orally, rectally, parenterally, intracistemally, intravaginally, intranasally, intraperitoneally, topically (as by powders, creams, ointments, or drops), bucally, or as an oral or nasal spray.

In various embodiments, liquid dosage forms for oral administration include pharmaceutically acceptable emulsions, microemulsions, solutions, suspensions, syrups, and elixirs. In addition to the active ingredients, the liquid dosage forms optionally further contain inert diluents or excipients.

The pH-responsive polymer compositions of the invention are used for intracellular delivery of a therapeutic agent (i.e., protein or peptide). The composition can be exposed to and contacted with a cell surface (e.g., via directed targeting) in a medium at a first pH. The composition is introduced into an endosomal membrane within the cell, for example, through endocytosis and, in some embodiments, through receptor mediated endocytosis. The endosomal membrane is destabilized (e.g., by the polymeric carrier, which is a membrane destabilizing polymer), thereby delivering the therapeutic agent (e.g., protein or peptide) to the cytosol of the cell.

In one aspect, the invention provides a method for delivering a protein or peptide antigen to cell's cytosol, comprising contacting a cell with a conjugate or a particle of the invention.

In another aspect, the invention provides a method for inducing a cytotoxic T-lymphocyte response, comprising contacting a cell with a conjugate or a particle of the invention.

In another aspect, the invention provides a method for providing tumor protection to a subject, comprising administering to a subject a therapeutically effective amount of the conjugate of a conjugate or a particle of the invention, wherein the immunotherapeutic agent is a protein or peptide cancer antigen. The term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition.

In another aspect, the invention provides a method for introducing a tumor-specific protein antigen to an antigen presenting cell to induce an immune response against the antigen and cells presenting the antigen, comprising contacting an antigen presenting cell with a conjugate or a particle of the invention. Representative antigen presenting cells include dendritic cells, macrophages, and B cells.

As noted above, in one aspect, the invention provides immunotherapeutic conjugates that include at least two components: (1) a polymeric carrier, a synthetic polymer consisting of hydrophobic alkyl groups and acidic carboxyl groups with polymer compositions chosen so that the carboxyl groups are ionized at physiological pH and (2) one or more immunotherapeutic agents covalently coupled thereto.

The polymers remain inactive in their ionized, hydrophilic state at pH 7.4. However, at the decreased pH levels typical of the endosome (pH 5.5-6.0), the carboxyl groups become protonated and the polymers transition to a state that is membrane destabilizing. This mechanism directly mimics that of viral and other pathogenic proteins, such as hemaglutinin and diphtheria toxin, that have evolved to enhance endosomal escape, and of synthetic fusogenic peptides such as GAL4 designed to imitate this behavior. The mechanistic utility of pathogenic proteins has demonstrated the importance of endosomal release in MHC1 presentation and CTL activation, but corresponding biological responses to the pathogenic proteins has limited their clinical translation.

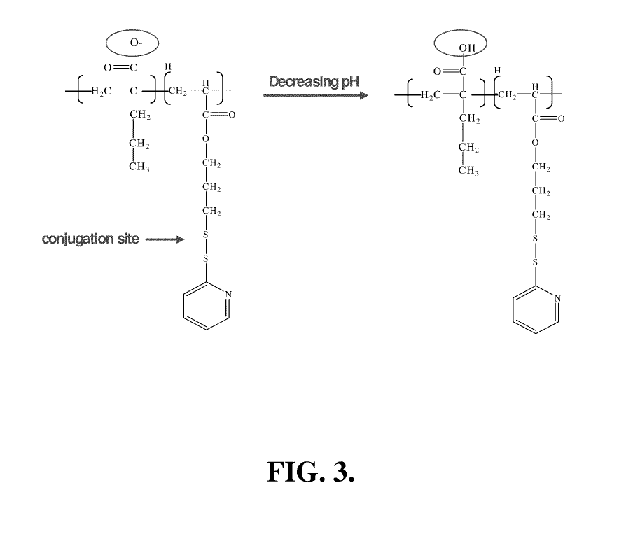

The present invention provides pH-responsive, membrane-destabilizing polymeric carriers for cytosolic protein antigen delivery. A representative polymeric carrier of the invention is illustrated in FIG. 3. Referring to FIG. 3, this poly(propylacrylic acid-co-pyridyldisulfide acrylate) (PPAA-PDSA) polymer is designed to conjugate a protein antigen by disulfide exchange between thiolated protein and the PDSA monomers. The resulting protein-polymer disulfide bond can be reduced by the cytosolic glutathione reduction system to leave the antigen free for subsequent proteolytic processing. The formulation of this carrier and its ability to enhance MHC1 presentation and CTL activation in protein immunotherapeutic strategies is described herein.

The following describes the synthesis of a representative polymer carrier of the invention (PPAA-PDSA) and its conjugation to a model protein (ovalbumin); the evaluation of the ability of this conjugate to enhance MHC1 presentation in macrophages; the uptake and intracellular accumulation of PPAA conjugates using radiolabeled ovalbumin; a particulate PPAA-ovalbumin formulation prepared by incorporating a cationic complexing agent, the uptake and intracellular accumulation of these particles, and the ability of the particles to increase MHC1 presentation of ovalbumin in a dendritic cell line; in vivo evaluation of both the soluble and particulate PPAA formulations in an ovalbumin-expressing tumor model in mice; and a study in which the ability of PPAA to deliver tumor antigenic MHC1 peptides to primary dendritic cells.

Conjugation of Poly(Propylacrylic Acid) Enhances the MHC-1 Presentation of Ovalbumin in a Macrophage Cell Line

One considerable barrier to effective immunotherapy strategies is delivery of the therapeutic antigen into the MHC-1 presentation pathway, accessed primarily in the cytosol, of antigen presenting cells. This pathway leads to activation of the potent cytotoxic T-lymphocytes, which are able to destroy diseased cells. This study utilizes a synthetic, pH-responsive carrier based on poly(propylacrylic acid) (PPAA) to enhance cytosolic delivery and subsequent MHC-1 presentation of the model protein antigen ovalbumin. Increased cytosolic delivery is achieved due to the membrane destabilizing action of PPAA in response to the acidic endosomal pH following cellular uptake of polymer-antigen conjugates. This system mimics the endosomal escape mechanism employed by viral and microbial agents to access the cytoplasm of host cells, while circumventing the toxicity and immunogenicity associated with these pathogenic agents. The carrier is designed to incorporate ovalbumin via a disulfide bond, which can then be reduced by the cytosolic glutathione reduction system to leave the antigen free for subsequent proteolytic processing.

Ovalbumin-PPAA conjugates are capable of pH-sensitive membrane disruption, as determined by a red blood cell hemolysis assay. These results suggest that the PPAA-antigen conjugates should be capable of escaping the endosome, allowing ovalbumin to access the MHC-1 pathway. This was then tested using a cytotoxic T-lymphocyte (CTL) activation assay. This model utilizes a CD8+ T-cell hybridoma that is activated upon T-cell receptor ligation to an MHC-1/ovalbumin peptide complex displayed from a model RAW macrophage cell. The PPAA-ovalbumin conjugates exhibited considerable increases in the MHC-1 presentation of ovalbumin, compared to negative controls: free ovalbumin, ovalbumin and PPAA mixed physically, and ovalbumin conjugated to a non membrane-active polymer, poly(methacrylic acid). Finally, toxicity of these carriers was evaluated in both the macrophage and T-cell lines and found to be negligible. Together, these results suggest PPAA-based delivery systems are well-suited for enhancing the efficacy of therapeutic protein vaccines.

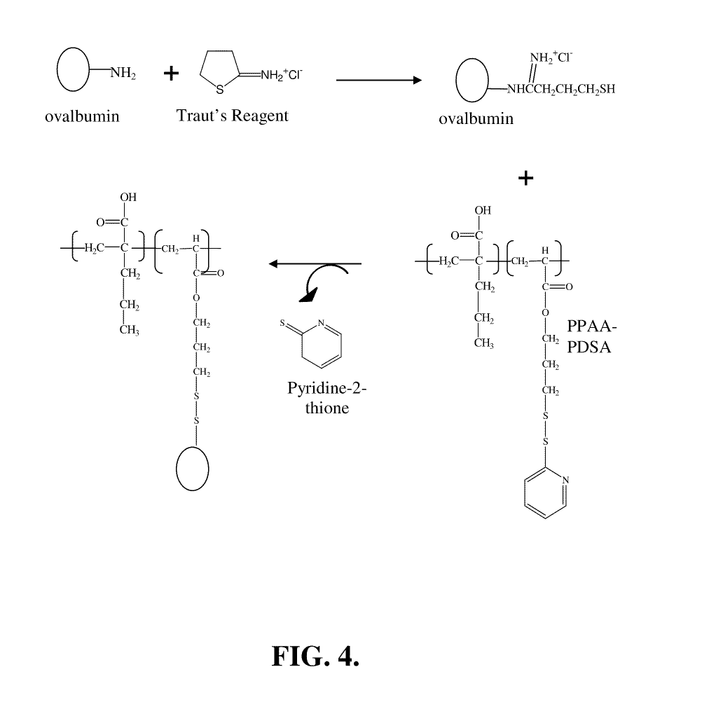

The conjugation scheme for attaching ovalbumin to PPAA-PDSA is described in Example 1 and shown in FIG. 4. The conjugates are formed through disulfide exchange of the ovalbumin thiol with the PDSA group in the polymer, and are designed to be reduced by glutathione in the cell cytoplasm, freeing the protein for processing and MHC-1 presentation.

The conjugation reaction highly favors polymer-protein combinations over polymer-polymer and protein-protein combinations. Pyridine-2-thione is a strong leaving group, making disulfide exchange between the thiol on the protein and the disulfide bond in PDSA much more likely than formation of a disulfide bond between two thiols on protein molecules. The polymer contains very few thiols to initiate disulfide exchange, as determined by measuring the initial A₃₄₃ of potentially dissociated PDSA in the polymer sample prior to conjugation. If such a reaction were to occur, the resulting polymer-polymer disulfide would likely be exchanged with a protein due to the higher concentration of protein thiols.

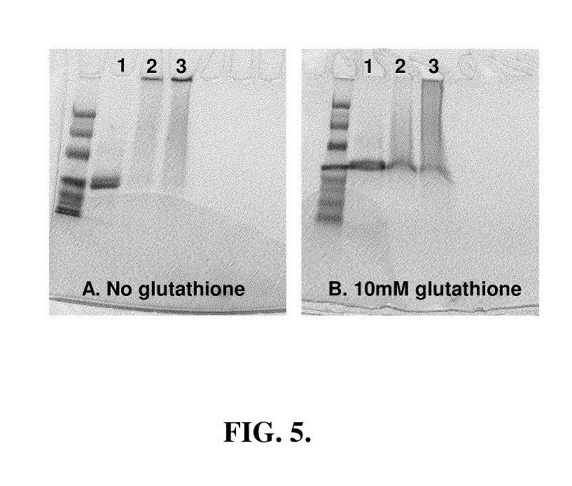

The glutathione reversibility of the conjugation is demonstrated in FIG. 5. This is an important feature as it allows the protein to be readily freed from the polymer carrier once it reaches the cytoplasm. The experiment was performed using a physiological glutathione concentration of 10 mM. However, the amount of glutathione able to reduce the polymer-protein disulfide bond is likely greater in the cytosol, due to continued regeneration of reactive glutathione by the enzyme glutathione reductase.

Red Blood Cell Hemolysis. Red blood cell hemolysis assays were performed to evaluate the pH-dependent membrane disruptive activity of the polymers and polymer-protein conjugates. Lysis of red blood cells demonstrates the ability of the polymer to disrupt lipid membranes and has been shown to correlate with endosome disruption. Membrane destabilization results from the hydrophilic to hydrophobic transition that occurs near the pKa of the PPAA polymer. At pH 7.4, the carboxyl groups making up the polymer backbone are present primarily in their ionized form, making the polymer hydrophilic. As the pH decreases, the carboxyl groups become protonated, and this resulting increase in hydrophobicity allows the polymer to destabilize the cell membrane, releasing hemoglobin.

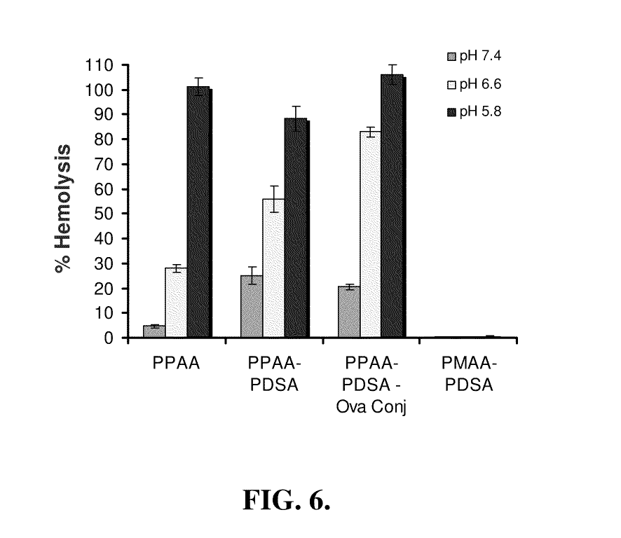

Hemolysis assays were conducted at three pH values (7.4, 6.6, and 5.8) in order to approximate physiological conditions as well those encountered in the early and late endosomes. Conjugate concentrations were adjusted to contain 5 μg/ml of polymer. The results of this assay are detailed in FIG. 6. As expected, the PPAA-PDSA polymer and conjugates were considerably more hemolytic at the lower pH characteristic of the endosome (pH 5.8) than at physiological pH: the PPAA polymer demonstrated 90% hemolysis at pH 5.8 and only 30% hemolysis at pH 7.4.

The PPAA-PDSA polymer shows slightly higher hemolysis at physiological pH than does a PPAA homopolymer, which consistently gave less than 10% hemolysis at pH 7.4 while exhibiting close to 100% hemolysis at pH 5.8. This increased hemolysis at pH 7.4 is likely due to the hydrophobicity added by the PDSA monomer, especially the pyridyl group. The increased hemolysis at pH 7.4 has not resulted in increased cytotoxicity at the concentrations and incubation times used, especially as many of these groups are released during the protein conjugation reaction. However, should this become a significant concern, excess pyridyl groups can be released using a thiol containing compound. The PPAA-ovalbumin conjugates have similar hemolysis profiles to the PPAA-PDSA polymer alone, indicating that the polymer retains its hemolytic activity when attached to a 40 kD hydrophilic protein. The retention of pH-sensitive hemolytic behavior in the conjugates is important because it indicates that the conjugates will be able to escape to the cytoplasm following endosomal uptake.

In contrast to the PPAA polymers, which become protonated at endosomal pH and are sufficiently hydrophobic to interact with lipid membranes, the PMAA polymer and conjugate were not hemolytic at any pH. This is because PMAA does not possess sufficient hydrophobic character to interact with membranes at any of the pH values tested. Since PMAA is similar in nature to PPAA but is not hemolytic, it is a potentially useful negative control polymer with which to compare the antigen delivery capabilities of PPAA.

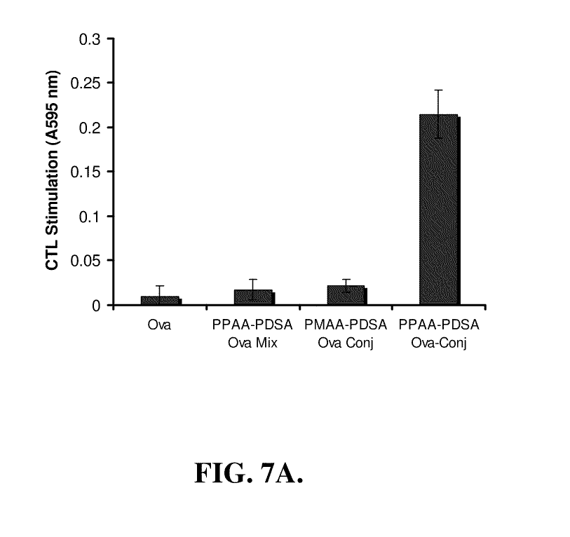

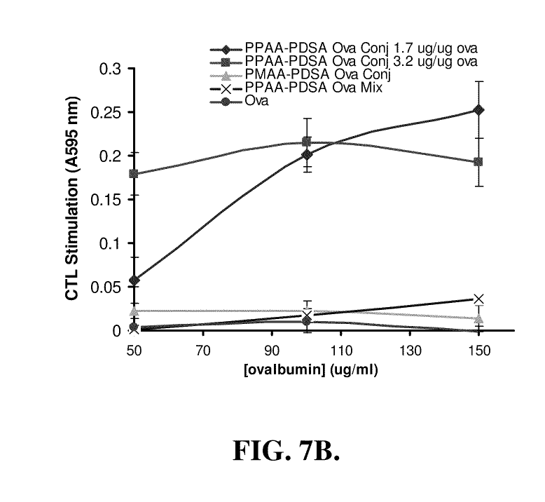

The ability of the polymer to enhance delivery of protein antigen from the endosome to the cytoplasm of APCs and into the MHC class I antigen presentation was evaluated using the LacZ class I presentation assay described in Example 1. The PPAA-PDSA-ovalbumin conjugate resulted in a strong 22-fold increase in MHC-I presentation and CTL activation compared to free ovalbumin (p=0.0001), as shown in FIG. 7A.

It can also be seen in this figure that the ovalbumin must be chemically attached to the PPAA in order to be effectively delivered into the MHC-1 pathway, as physical mixtures showed only background CTL activation levels (p=0.20 compared to free ovalbumin). Conjugation to PPAA also resulted in much greater CTL activation than did conjugation to the non membrane-disruptive polymer, PMAA (p=0.0002, 11-fold increase in CTL activation The PMAA-ovalbumin conjugates did not significantly enhance CTL activation over delivery of free ovalbumin (p=0.72.) These results correlate the membrane destabilizing activity of the polymer to the level of MHC-1 presentation and CTL activation, and suggest that the increase in presentation is not solely the result of increased cellular uptake due to the larger size of the conjugate compared to free ovalbumin.

Additionally, PPAA conjugation increases the CTL activation in a dose-dependent manner, whereas higher concentrations of control samples do not result in significant increases in CTL activation (p<0.2) (FIG. 7B). Increasing the amount of PPAA in the conjugate further exaggerates this effect: when the weight ratio of polymer:protein is doubled from 1.7 to 3.2 μg polymer per μg protein, CTL activation increases, becoming maximized at a lower ovalbumin concentration. The overall increases in CTL activation provided by conjugation to PPAA in comparison with the various control groups are summarized in Table 1.

The PPAA carrier performs well when compared to other protein delivery systems which have been evaluated using the B3Z CTL activation assay. Acid-degradable particles used to deliver ovalbumin have resulted in maximal A₅₉₅ values (the measure of CTL activation) in the range of 0.25-0.4, which is similar to that reported in our study, and PLGA particles have been shown to provide a 5-fold increase in CTL activation over delivery of ovalbumin alone. While direct comparisons are not possible due to the different carrier architectures (soluble vs. particulate), and differences in experimental conditions and data presentation, it is useful to put the PPAA carrier into the context of other protein delivery systems.

CTL activation increases observed for PPAA-PDSA-Ovalbumin conjugates are summarized in Table 1. The fold increase in CTL activation is given for the test sample, listed in the first column, when compared to the control sample listed in the second column. Furthermore, p-values resulting from ANOVA statistical analysis are provided in the final column. p-values>0.05 are considered statistically insignificant. This table clearly shows the advantages in CTL activation provided by conjugation to PPAA.

TABLE 1
Summary of CTL activation increases observed for
PPAA-PDSA-Ovalbumin conjugates.

| | Sample 2: | Fold Increase in | |
|---|---|---|---|
| Sample 1: | Control | CTL Activation: | ANOVA |
| Test Sample | Sample | Sample 1/Sample 2 | p-value |
| PPAA-PDSA-Ova Conj. | Ovalbumin | 22X | 0.0001 |
| PPAA-PDSA-Ova Conj. | PMAA- | 11X | 0.0002 |
| | PDSA-Ova | | |
| | Conj. | | |
| PPAA-PDSA-Ova Conj. | PPAA- | 12X | 0.0003 |
| | PDSA Ova | | |
| | Mix | | |
| PPAA-PDSA Ova Mix | Ovalbumin | 1.9X | 0.20 |
| PMAA-PDSA-Ova Conj. | Ovalbumin | 2.2X | 0.72 |

Polymer Composition Cytotoxicity. The cytotoxicity of the PPAA-PDSA and PMAA-PDSA polymers and conjugates was tested in both cell lines used in this study: RAW macrophages and B3Z T-cells. Concentrations up to 300 μg/ml, twice the concentration used in the MHC-I presentation assay, were tested using the LDH assay. This assay allows colorimetric measurement of

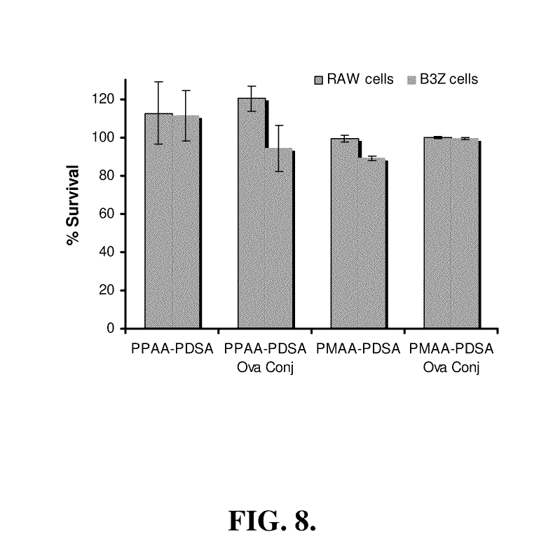

LDH activity in the supernatant, which correlates to the proportion of dead or damaged cells. Cell survival was calculated by comparing the polymer-treated cells with untreated cells and with cells lysed with 1% Triton X-100. It can be seen in FIG. 8 that none of the PPAA-PDSA or PMAA-PDSA polymers or conjugates exhibit considerable toxicity in either cell type at a concentration of 300 μg/ml.

A representative pH-sensitive, membrane-disruptive polymer, poly(propylacrylic acid) (PPAA), was synthesized and used to enhance the cytoplasmic delivery and MHC-I presentation of a model protein antigen, ovalbumin. The protein was derivatized to have a thiol group, which was reacted with the pendant PDSA disulfide bond on the polymer to yield a polymer-protein conjugate linked by a disulfide bond. This polymer-S—S-protein linkage was designed to allow release of the protein by glutathione reduction in the cytoplasm. The polymer exhibited low toxicity in vitro and retained its membrane-disruptive capabilities after attachment to a hydrophilic protein, as indicated by a red blood cell hemolysis assay. Conjugation to the PPAA polymer was shown to significantly enhance the MHC-I presentation of ovalbumin and subsequent CTL activation. This effect is a result of increased cytoplasmic delivery of ovalbumin, which occurs due to destabilization of the endosomal membrane by PPAA. These results suggest that the intracellular pharmacokinetic step of vesicular release is a limiting barrier to subsequent protein processing and MHC-1 presentation. This system thus shows promise for protein vaccine strategies against cancer and viruses, and is also applicable to any technique requiring improved delivery of a protein cargo to the cytoplasm of a cell.

Cellular Uptake and Exocytosis Profiles of Poly(Propylacrylic Acid)-Ovalbumin Conjugates

In order to further elucidate the enhanced CTL activation provided by the PPAA carrier and investigate the carrier's cytosolic delivery capabilities, the cellular uptake and exocytosis of radiolabeled ovalbumin with and without the carrier was evaluated. Several methods have been employed for conducting similar studies on intracellular trafficking of molecules. For example, fluorescently labeled molecules are followed using either confocal microscopy or flow cytometry, and these studies often employ lysosomal, nuclear, or outer membrane fluorescent markers to more specifically determine the molecule's localization. Radiolabeled compounds have also been employed to study cellular trafficking, sometimes in combination with subcellular fractionation techniques to obtain detailed localization information. ¹⁴C radiolabels were chosen for this study due to their high sensitivity and stability.

Cellular uptake and exocytosis of ¹⁴C-labeled ovalbumin was quantified in a RAW macrophage cell line. Delivery of ovalbumin conjugated to the endosome-disruptive PPAA carrier was compared to delivery of free ovalbumin, a physical mixture of PPAA and ovalbumin, and ovalbumin conjugated to non-active PMAA. The overall uptake of ¹⁴C-ovalbumin was measured after various incubation times by removing un-internalized ovalbumin and lysing the cells to measure the amount of intracellular ovalbumin. Enhanced intracellular accumulation of the PPAA-ovalbumin conjugate was observed. This can likely be attributed to continued escape of the PPAA-ovalbumin into the cytosol, whereas the unconjugated or PMAA-conjugated ovalbumin remains in the endo/lysosomal compartment, where it can be exocytosed from the cell.

To further investigate this hypothesis, the experiment was performed with several adjustments to specifically address exocytosis of these compounds, utilizing a method similar to those employed previously for the study of exocytosis of radiolabeled sucrose in guinea pig alveolar macrophages, hapten-protein conjugates in murine macrophages, and PLGA nanoparticles in vascular smooth muscle cells. The exocytosis of ¹⁴C-ovalbumin was measured by quantifying the amount of radioactivity re-appearing in the cell supernatant after removal of un-internalized ovalbumin. The PPAA-ovalbumin conjugates showed decreased exocytosis and increased intracellular accumulation compared to controls, which correlates very well with the enhancement in MHC-1 presentation and CTL activation observed in the previous study.

Radiolabeled conjugates were synthesized and were of similar sizes to the non-radioactive counterparts described above. See Example 2. The ¹⁴C-labeled PPAA conjugates had M_{w}=140 kD, Mₙ=40 kD, PDI=3.5 and the PMAA conjugate had M_{w}=120 kD, Mₙ=50 kD, PDI=2.4. The PPAA and PMAA conjugates were found to be 34 and 40 wt. % ovalbumin, or 1.9 μg polymer per μg protein and 1.5 μg polymer per μg protein, respectively.

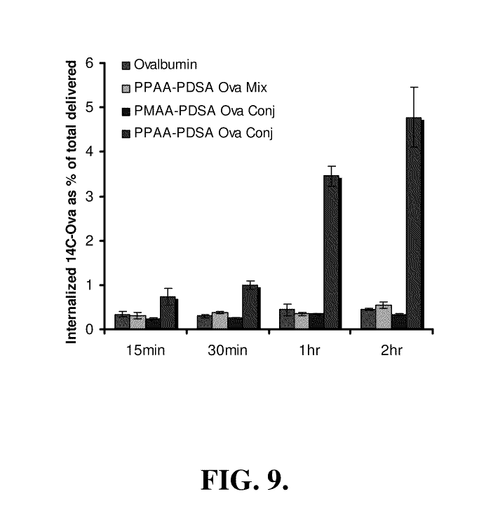

In order to further explore the intracellular release provided by PPAA conjugation, the cellular uptake and exocytosis of ¹⁴C-ovalbumin was studied in RAW macrophages. The intracellular accumulation of ¹⁴C-ovalbumin over increasing incubation times was measured for PPAA-ovalbumin conjugates as well as negative controls: free ova, PPAA+ova physical mixtures, and PMAA-ova conjugates. The enhanced intracellular accumulation of the PPAA-ova conjugate is demonstrated in FIG. 9. After a 15-min incubation, the amount of PPAA-conjugated ovalbumin inside the cell was already twice that of the control groups. Between 15 min and 2 hrs, the amount of intracellular PPAA-ovalbumin increased from 0.75% of the total amount delivered to 4.8%, whereas the control groups only increased from around 0.35% of the total amount delivered to 0.55%. These differences in cellular accumulation are likely due to continued escape of the PPAA-ovalbumin into the cytosol, whereas unconjugated or PMAA-conjugated ovalbumin remains in the endo/lysosomal compartment where it can be exocytosed.

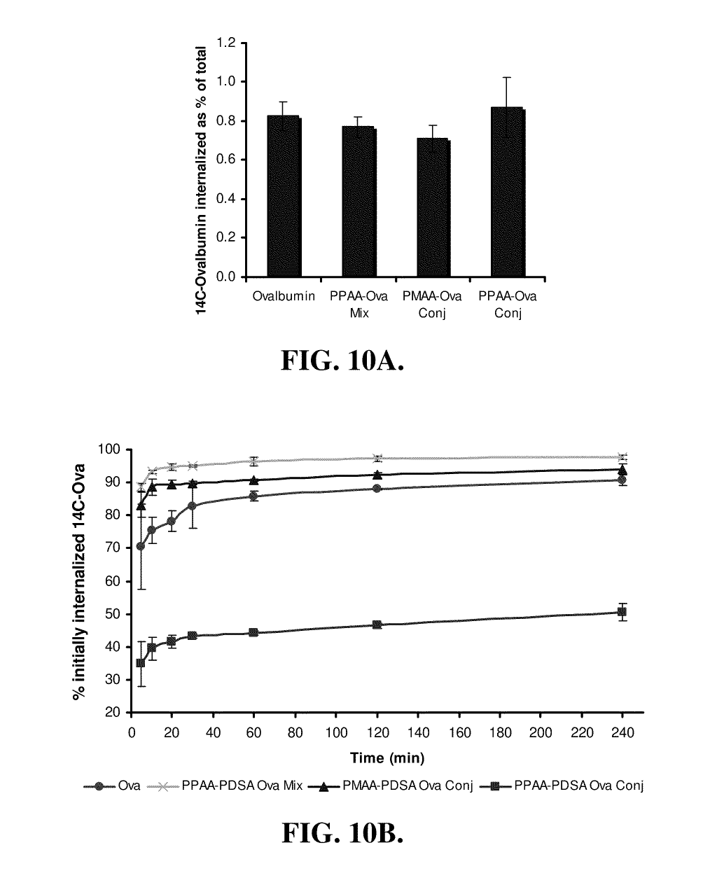

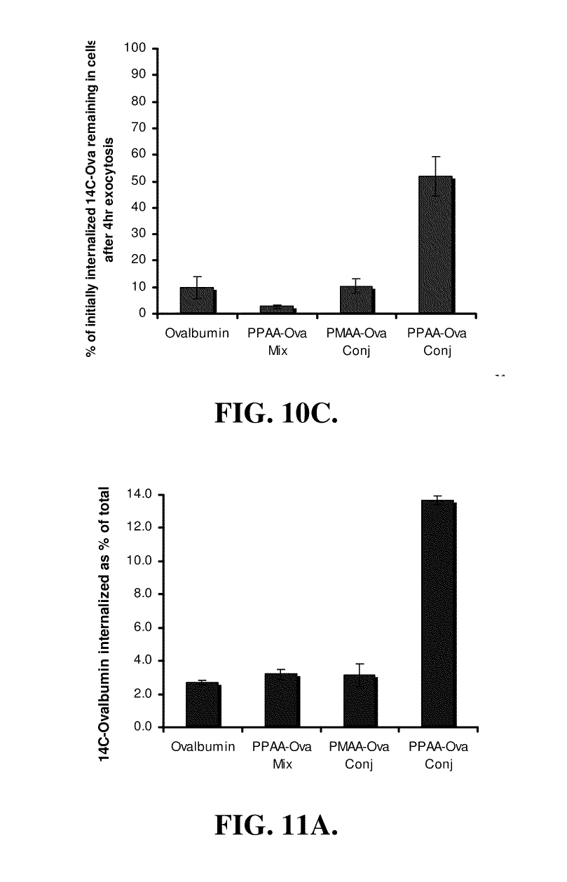

To investigate this hypothesis, the experiment was repeated with several adjustments to specifically address exocytosis of these compounds. The samples were incubated with RAW macrophages for 1 min, then any un-internalized sample was removed by washing the cells 2× with media. Fresh media was applied at regular time intervals from 5 min to 4 hrs, followed by lysis of the cells. The amount of radioactivity which reappeared in the supernatant at each time interval, as well as the radioactivity remaining in the cell lysate, was measured. This method is similar methods previously employed for the study of exocytosis of radiolabeled sucrose in guinea pig alveolar macrophages, hapten-protein conjugates in murine macrophages, and PLGA nanoparticles in vascular smooth muscle cells. Following the short 1-min incubation time, the initial amount of ¹⁴C-ovalbumin taken up by the cells was statistically similar for all the sample groups (ANOVA p-value=0.66), and was approximately 0.8% of the total amount delivered (FIG. 10A).

However, less PPAA-conjugated ovalbumin was exocytosed compared to the control groups, which is detailed in the exocytosis profiles shown in FIG. 10B. This resulted in greater accumulation of the PPAA-ovalbumin inside the cells, even after 4 hours of exocytosis (FIG. 10C). This figure shows that 52% of the internalized PPAA-ovalbumin was still inside the cells at the end of the experiment, whereas less than 10% of the ovalbumin remained for all the control samples, including the non membrane-active PMAA conjugate. This strongly supports the hypothesis that PPAA-ovalbumin accumulates in the cell as a result of decreased exocytosis. This effect is expected based on the ability of the PPAA polymer to disrupt the endosomal membrane and deliver the ovalbumin to the cytosol before exocytosis can occur.

These results further support the assertion that increased MHC-1 presentation and CTL activation is afforded by PPAA conjugation due to enhanced cytosolic delivery of ovalbumin, and is not solely a result of increased compound size. It can be noted that for all samples, reappearance of the radiolabeled ovalbumin in the supernatant was observed as early as 5 min after the cells were washed, and the majority of exocytosis occurred in the first 30 min, a timescale which is in accordance with previous exocytosis studies. Due to the negative charge of both the PPAA and PMAA polymers at physiological pH, it is highly unlikely that these results are affected by compounds sticking to and subsequently releasing from the cell's negatively charged outer membrane.

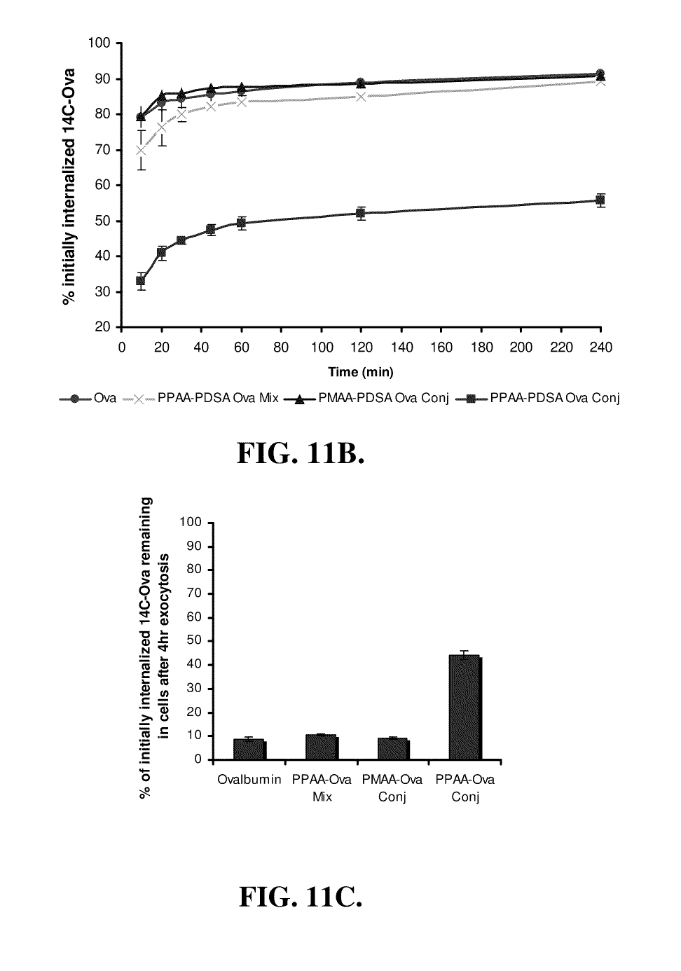

In order to verify the trends recorded in this experiment, and to reduce variability, the experiment was repeated using a longer 15-min initial incubation time of the radiolabeled compounds with the cells. The preferential intracellular accumulation of PPAA-conjugated ovalbumin can already be seen in the initial cell uptake at this incubation time (14% for PPAA-ovalbumin vs. 3% for control samples, FIG. 11A). However, after normalizing to the amount of ¹⁴C-ovalbumin internalized by the cells at the start of the exocytosis measurements, the rates of exocytosis (FIG. 11B) and the amounts of ovalbumin remaining in the cells at the end of the experiment (44% for PPAA-ovalbumin vs. 10% for control samples, (FIG. 11c) are shown to enforce the trends observed for the 1 min uptake time.

Cellular uptake and exocytosis of ¹⁴C-labeled ovalbumin was quantified in a RAW macrophage cell line. Ovalbumin conjugated to PPAA showed decreased exocytosis and increased intracellular accumulation compared to controls, which correlates very well with the enhancement in MHC-1 presentation and CTL activation observed. This can likely be attributed to continued escape of the PPAA-ovalbumin into the cytosol, whereas the unconjugated or PMAA-conjugated ovalbumin remains in the endo/lysosomal compartment, where it can be exocytosed from the cell.

Formulation and In Vitro Evaluation of Poly(Propylacrylic Acid)-Ovalbumin/Poly(dimethylaminoethylmethacrylate) Ionic Particles

Several studies have shown that particulate substances undergo increased uptake and cross presentation by antigen presenting cells than do soluble substances. In order to investigate whether a particulate formulation of our PPAA carrier would result in greater delivery efficiency, poly(dimethyl amino ethyl methacrylate) (pDMAEMA) was employed as a cationic complexing agent, which resulted in the formation of ionic particles 100-300 nm in size when combined with the anionic PPAA-ovalbumin conjugate. Cellular uptake and exocytosis of these particles to their soluble counterparts in a RAW macrophage cell line, and it was found that the particulate formulation was indeed advantageous. The ability of the carriers to enhance MHC1 presentation of ovalbumin was then evaluated in another cell line, the mouse dendritic cell line DC2.4. This cell line was employed as it was desired to test the carrier in a more dendritic-like cell line. The DC2.4 line was derived from murine bone marrow cells transfected with myc and raf oncogenes as well as the granulocyte macrophage colony stimulating factor (GM-CSF) gene. These cells possess the highly phagocytic nature of immature DCs, as well as the MHC presentation and expression of other surface markers characteristic of mature DCs. This line can be used in coordination with the B3Z CTL hybridoma, which produces B-galactosidase when activated, and has been used in previous studies for the evaluation of PLGA-based vaccine delivery systems. It was found that the PPAA particles resulted in substantially greater MHC1 presentation of ovalbumin than did the soluble conjugates, which indicates that either the uptake or cross-presentation, or both, are increased for particulate carriers in these cells.

The PPAA-PDSA and PMAA-PDSA polymers described above were used to form the ovalbumin conjugates. These conjugates were of a similar size to those used described above: M_{w}=118 kD, Mₙ=44 kD, PDI=2.7 for the PPAA-PDSA-ovalbumin conjugate and M_{w}=120 kD, Mₙ=55 kD, PDI=2.3 for the PMAA-PDSA-ovalbumin conjugate. GPC analysis of pDMAEMA in DMF using PMMA standards gave M_{w}=9.4 kD, Mₙ=7.8 kD, PDI=1.2.

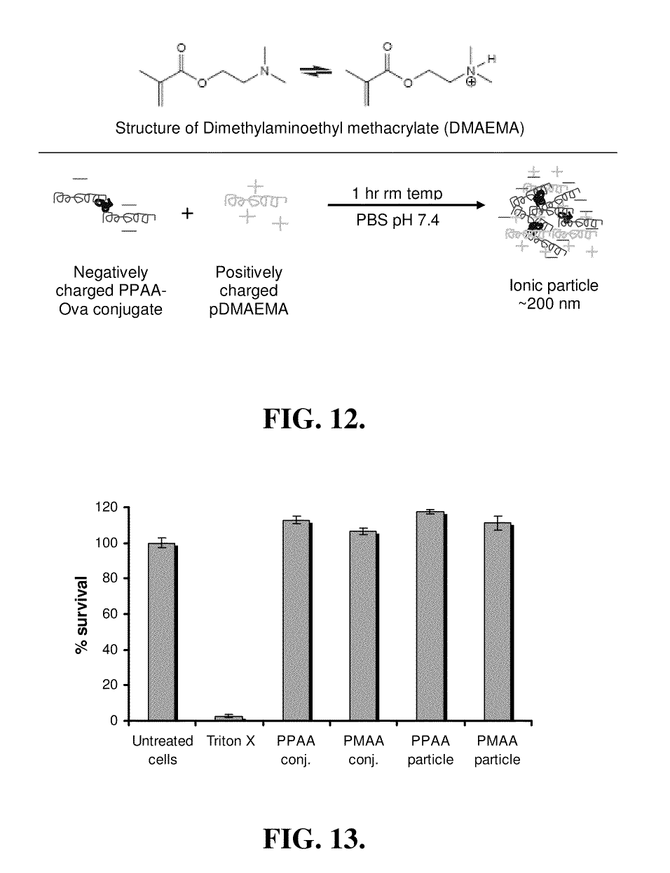

The particle formulation process is described in Example 3 and shown schematically in FIG. 12. The process proved to be very reproducible, and representative particle sizes and zeta potentials are given in Table 2 for several −/+charge ratios. All particle sizes are reported as the number average diameter. The charge ratios shown are theoretical, calculated from the amounts and sizes of the negatively charged PPAA and positively charged pDMAEMA and their expected degrees of ionization at pH 7.4. It can be seen that −/+charge ratios between 60:1 and 15:1 give particles in the target range of 100-300 nm. Charge ratios near 1:1 result in the formation of large aggregates. The zeta potentials were strongly negative for all the charge ratios. Since the zeta potential is a measure of the charge on the particle surface, it is not necessarily expected to increase with an increase in + charge in this range, as the overall ratio is still largely negative. Positively charged particles were not used, as the amount of pDMAEMA necessary to shift the net particle charge to positive was very great and resulted in excessive toxicity and adherence to cells in preliminary in vitro studies.

TABLE 2
Representative sizes of PPAA/pDMAEMA ionic
particles as measured by dynamic light scattering.

| −/+ charge | Particle diameter | | Zeta |
|---|---|---|---|
| ratio | (number avg) | Stdev. | potential (mV) |
| 60:1 | 72 nm | +/−15 | −39.8 |
| 40:1 | 125 nm | +/−28 | −35.4 |
| 20:1 | 178 nm | +/−45 | −32.6 |
| 15:1 | 250 nm | +/−65 | −28.4 |
| 10:1 | 288 nm | +/−67 | −20.2 |
| 5:1 | 1990 nm | +/−470 | −21.3 |
| 2:1 | Large aggregates | — | NA |
| 1:1 | Large aggregates | — | NA |

The toxicity of PPAA-Ova and PMAA-Ova conjugates and their corresponding pDMAEMA particles was tested after a 24-hour incubation with RAW macrophages, using the Alamar Blue assay. This assay utilizes a fluorescent redox indicator to quantify cell growth and metabolism, and can therefore be used to measure cytotoxicity. The particles were shown to cause no appreciable toxicity at polymer concentrations up to 750 μg/ml, 5× that used in the cellular uptake and MHC-1 presentation assays (FIG. 13). The slight increase in cell viability observed in the figure may be a result of the conjugates containing ovalbumin, which the cells could use as a nutrient. This result is expected based on previous results showing PPAA and PMAA polymers to be nontoxic in an LDH cytoxicity assay (section 2.3.4). In this experiment, the particles were established as nontoxic, which is important given their larger size and different architecture compared to the soluble polymers.

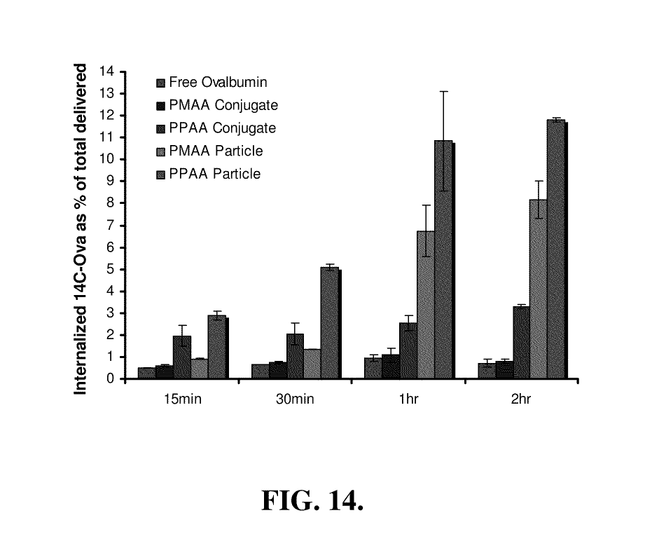

The uptake and exocytosis of the PPAA-Ova/pDMAEMA particles was compared to the soluble conjugates in a RAW macrophage cell line as described in Example 3. The amount of internalized ¹⁴C-Ova was measured after incubation times of 15 min, 30 min, 1 hr, and 2 hr, and the results are depicted in FIG. 14. It can be seen that the PPAA particle does indeed result in increased intracellular accumulation compared to the soluble PPAA conjugate. This effect becomes more pronounced with increasing uptake time: uptake of the PPAA particle is 1.5× that of the PPAA conjugate after 15 min of uptake, and increases to 3.6× after 2 hrs. It can also be seen that the non-membrane active PMAA particle shows increased uptake over the soluble PPAA conjugate, mainly at the longer uptake times. This suggests that particulates accumulate more efficiently than soluble substances in this cell type, and that this effect is not dependent on membrane-disruptive activity of the polymer. This may be due to a more efficient uptake mechanism for particulate substances through phagocytosis, which has been report to be very highly efficient. Also, a different, lengthier mechanism is implicated since the particles show preferential accumulation primarily at the longer incubation times (>1 hr), but not the shorter incubation times (<30 min). An intrinsic cytosolic delivery pathway has been suggested for uptake by the processes of phagocytosis, and it has been shown that latex beads taken up by phagocytosis start to appear in the cytosol 1-2 hours after exposure to macrophages.

If there are indeed different uptake efficiencies or uptake mechanisms involved for the particulate vs. soluble conjugates, it may be difficult to completely separate the particle-induced change in uptake/processing from PPAA's endosomal release effects. It is important to note that the intracellular accumulation of the PPAA particle is 45% greater than that of the PMAA particle of the same size, even at the 2 hr timepoint. The PPAA particles, therefore, may benefit from a combined effect of increased uptake/cytosolic delivery due to their particulate nature and enhanced cytosolic delivery due to endosomal escape provided by the polymer. While soluble and particulate carriers are both beneficial, depending on their application, a pH-sensitive particulate carrier may prove to be the most optimized vehicle for immunotherapeutic strategies.

To further explore the uptake and retention of these soluble vs. particulate substances, the exocytosis of the substances was followed as described in Example 3. The samples were incubated with RAW macrophages for 15 min, then any un-internalized sample was removed by washing the cells 2× with media. Fresh media was applied at regular time intervals from 5 min to 4 hrs, followed by lysis of the cells. The amount of radioactivity which reappeared in the supernatant at each time interval, as well as the radioactivity remaining in the cell lysate, was measured. Increased intracellular accumulation and decreased exocytosis was observed for PPAA particles and soluble conjugates, and to a lesser extent for PMAA particles.

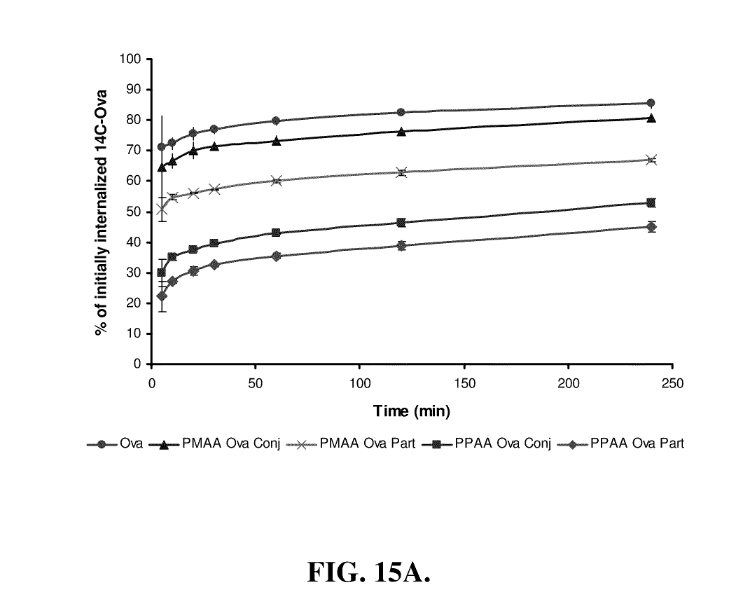

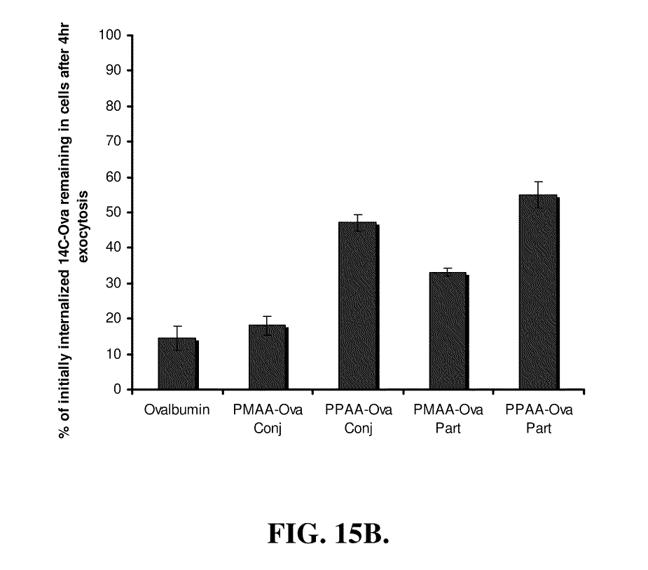

The exocytosis profiles for all samples are shown in FIG. 15A, with the amount of ¹⁴C-ovalbumin remaining in the cells at the conclusion of the exocytosis study summarized in FIG. 15B. Most exocytosis occurs in the first 5-10 minutes. It can be seen in FIG. 15B that both the PPAA particle and conjugate show greater retention/reduced exocytosis than does the PMAA particle (p<1×10⁻⁶). After 4 hours of exocytosis, 55% of the internalized PPAA particle and 47% of the internalized PPAA conjugate, respectively, remain inside the cell, whereas only 33% of the PMAA particle remains inside the cell. The amount is even lower for the free ovalbumin (14%) and soluble PMAA conjugate (18%).

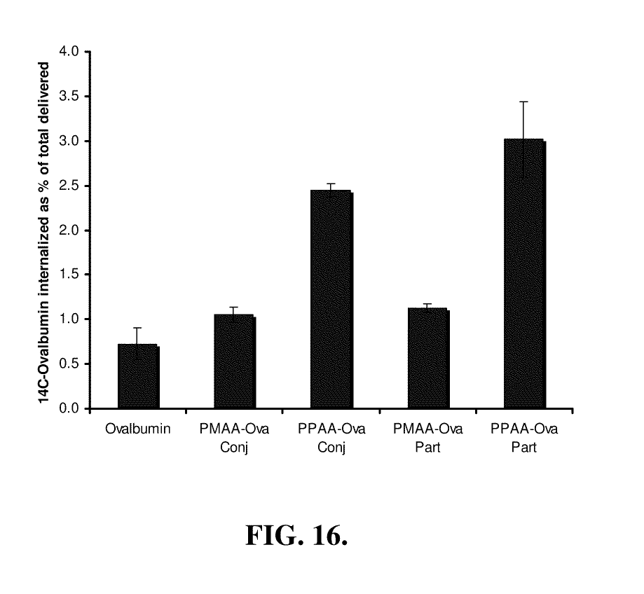

The amount of initially internalized ovalbumin at the beginning of this exocytosis study is greater for the PPAA conjugate and particle than the other samples (FIG. 16). This is in agreement with the studies discussed in section 3.3.3 showing decreased exocytosis of PPAA-ova starting at very early timepoints, which even by 15 min can result in a noticeable excess of PPAA-conjugated ovalbumin in the cell.

When a very short 1 min uptake time was used, the initial amount of ¹⁴C-ovalbumin taken up by the cells was statistically similar for all the sample groups (p=0.66). Furthermore, at the short 1 min uptake time, the non membrane-active PMAA particle did not result in increased intracellular retention (after 4 hrs exocytosis) compared to the free ovalbumin and PMAA soluble conjugate controls (p>0.39), whereas the PPAA was able to provide enhanced cellular retention of internalized substances in both the soluble and particulate form (p<0.03) (data not shown). The difference in retention observed with the PMAA particle at the 1 min and 15 min uptake times could be due to a longer uptake/processing time required for particulate matter.

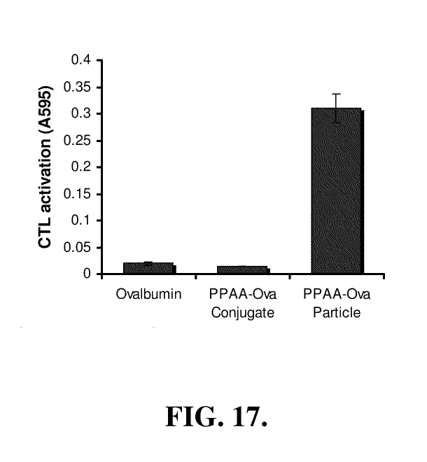

The antigen presentation of ovalbumin delivered in PPAA ionic particles was evaluated by the lacZ antigen presentation assay as described in Example 3 using the dendritic cell line DC2.4 as the antigen presenting cells. It was found that the particles did enhance MHC-1 presentation/CTL activation when compared to the free ovalbumin and to the soluble PPAA conjugates, by about 15-fold (insert pvalue), as shown in FIG. 17. Interestingly, the soluble conjugates did not result in significantly increased presentation over delivery of free ovalbumin (insert pvalue). This may be due to the nature of this cell line, which has consistently shown increased phagocytosis and cross-presentation of particulate substances compared to soluble substances for other materials as well. The results of this study therefore indicate that the PPAA-ovalbumin particles will be promising carriers for the delivery of protein therapeutics in an in vivo vaccination model.

In this study, the PPAA conjugates were incorporated into a particulate formulation by addition of an ionic complexing agent, pDMAEMA. When the uptake and exocytosis of the particles was evaluated in RAW macrophages, the intracellular accumulation of PPAA particles was found to be greater than both the soluble PPAA conjugates and the PMAA control particles. This suggests that they may benefit from a combined effect of increased uptake/cytosolic delivery due to their particulate nature and enhanced cytosolic delivery due to endosomal escape provided by the polymer. The PPAA particles provided enhanced MHC-1 presentation of ovalbumin in a dendritic cell line, compared to both free ovalbumin and the soluble PPAA conjugate, and exhibited no toxicity in vitro. These combined results indicate that the PPAA particles, in addition to the soluble conjugates, show promise for in vivo vaccination applications.

Delivery of Poly(Propylacrylic Acid)-Ovalbumin Vaccines Provides Enhanced Protection Against Ovalbumin Expressing Tumors in Mice

Because PPAA is a promising carrier for protein vaccines in in vitro studies, in both its soluble and particulate forms, its therapeutic effect was evaluated in an in vivo model. A previously established murine ovalbumin tumor model was employed. This model utilizes a tumor cell line, E.G7-OVA, which is derived from the C57B1/6 mouse EL4 thymoma cell line by electroporation with ovalbumin plasmid DNA. Since the tumor cells are derived from the inbred C57B1/6 mouse strain, they can be injected into fully immunocompetent C57B1/6 mice to form ovalbumin expressing tumors. The tumor cells produce and process cytosolic ovalbumin, presenting peptides on MHC1 molecules. Therefore, ovalbumin can be delivered as a protein antigen in immunotherapeutic strategies. This tumor model can provide valuable insight into carrier performance and has been used by researchers to evaluate delivery systems utilizing IL-2-containing liposomes, acid degradable particles, mannose-coated liposomes, viral translocation motifs, cholera toxin, and various adjuvants such as α-galactosylceramide, poly (I:C)/anti CD-40, and imiquimod.

The therapeutic effect was evaluated in an in vivo model as described in Example 4. Vaccines were injected 7 days prior to tumor cell injection. Both soluble and particulate PPAA formulations were evaluated, using PBS, free ovalbumin, and PMAA soluble and particulate carriers as controls. In order to further explore the immune response, blood was drawn from all mice and used to determine anti-ovalbumin IgG antibody titer by ELISA. Finally, the number of ovalbumin reactive CD8+ T cells present in the spleens of the mice was measured by flow cytometry using MHC-1/SIINFEKL specific tetramers. These tetramers are fluorescently labeled and bind to the T-Cell Receptor of CD8+ T-cells that are reactive to ovalbumin. The results of the in vivo analysis showed that both the soluble and particulate PPAA conjugates provided substantial tumor protection compared to delivery of ovalbumin alone. Control mice which received only PBS for the vaccine injection began to grow tumors 5 days after tumor cell injection. Mice vaccinated with free ovalbumin began to grow tumors 8 days after tumor cell injection and showed only slightly slower growth compared to the PBS mice. All PBS and ovalbumin-injected mice had to be euthanized by 19 days after tumor injection, as the tumors exceeded a volume of 2 cm³. The mice receiving PPAA-ovalbumin particulate formulations remained tumor free 4 weeks after tumor cell injection, and only 1 mouse in the PPAA-ovalbumin soluble group had begun to develop a tumor. These carriers were shown to result in increases in both the antibody and CD8+ response, compared to controls. These preliminary results suggest that PPAA is a promising carrier for in vivo immunotherapy strategies.

The samples used in the in vivo studies are characterized in Table 3 below. The particle sizes reported are the number average. The PPAA-PDSA and PMAA-PDSA polymers used to form the samples are described in the examples.

TABLE 3
Characteristics of the PPAA and PMAA conjugates and particles
utilized in the in vivo studies.

| | Conjugate | | | |
|---|---|---|---|---|
| | Size (GPC) | | | Particle Size (DLS) |
| | M_{w} | Mₙ | PDI | size +/− stdev. |
| PPAA-Ova | 124 kD | 43 kD | 2.9 | 128 nm +/− 32 nm |
| PMAA-Ova | 130 kD | 54 kD | 2.4 | 170 nm +/− 34 nm |

The in vivo efficacy of PPAA as a vaccine carrier was evaluated using a murine ovalbumin tumor model. This model utilizes the E.G7-OVA tumor cell line, a derivative of the EL4 thymoma line which has been transfected with the ovalbumin gene so that the tumors formed present ovalbumin. These cells were derived from the same mouse strain used in the study, C57B1/6, which ensures immunocompatibility of the tumor cells themselves. This model has been previously employed for the characterization of PLGA and acid-degradable particulate systems as well as to evaluate various adjuvants and has proven a very informative indicator of vaccine function.

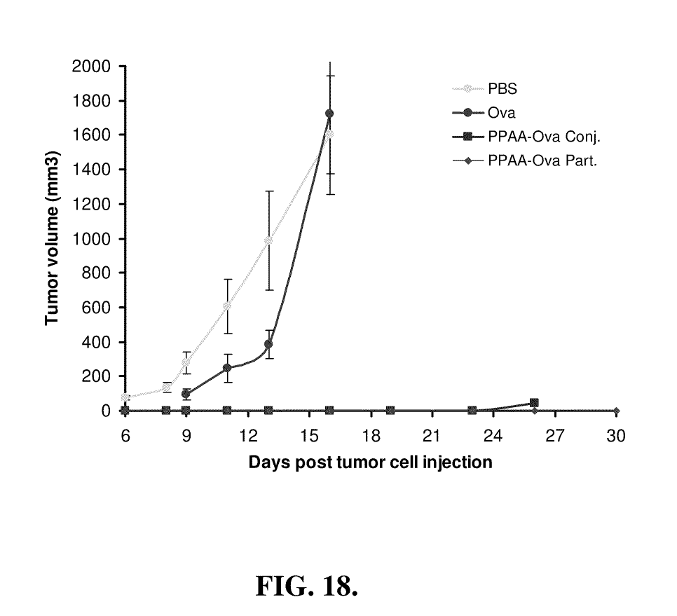

The PPAA-ovalbumin and control vaccines were delivered subcutaneously to the mouse's right flank 7 days prior to the injection of the tumor cells subcutaneously to the left flank. This causes formation of a tumor just beneath the skin which can be measured to calculate the tumor volume. Tumor growth was monitored for 4 weeks. Both the soluble and particulate PPAA carriers provided excellent tumor protection, as evidenced by the tumor growth plots shown in FIG. 18.

Control mice which received only PBS for the vaccine injection began to grow tumors 5 days after tumor cell injection. Mice vaccinated with free ovalbumin began to grow tumors 8 days after tumor cell injection and showed only slightly slower growth compared to the PBS mice. All PBS and ovalbumin-injected mice had to be euthanized by 19 days after tumor injection, as the tumors exceeded a volume of 2 cm³. Statistical analysis performed on the tumor volumes at various timepoints show that the tumor sizes were not significantly different for the PBS vs. free ova mice (insert table of pvalues). After 4 weeks, all mice receiving the PPAA-ovalbumin particle vaccine remained tumor free and only 1 mouse which received the PPAA-ovalbumin soluble conjugate had begun to develop a tumor. There was not an appreciable difference between the soluble and particulate formulations.

The tumor protection provided by the PPAA carrier is very strong when placed in the context of other vaccine systems utilizing this model. Ovalbumin co-delivered with the adjuvant IL-12p40 provided protection for 9 days, ovalbumin delivered with IL-2 exosomes provided protection for 22 days, and ovalbumin delivered via IL-2-containing liposomes provided protection for 45 days. The results indicate that the PPAA carrier can stimulate an immune response capable of providing tumor protection.

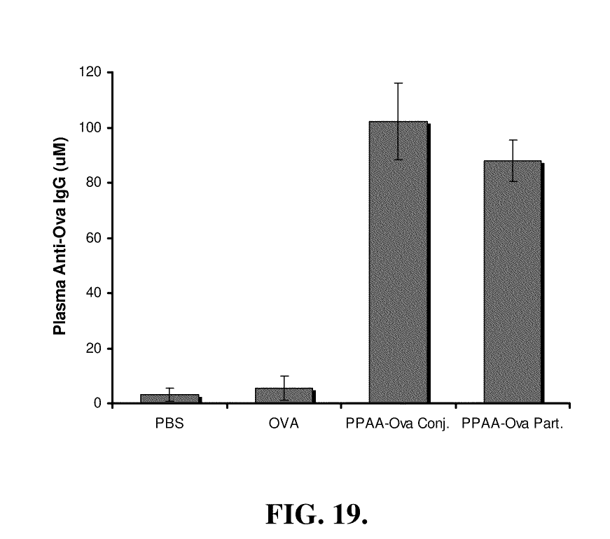

In order to determine the role of the humoral immune response in the tumor protection observed for PPAA-ova vaccinated mice, blood was drawn from all mice 20 days after vaccination and the anti-ova IgG titer was measured by ELISA. FIG. 19 shows the average antibody concentration per group, as determined by an anti-ova IgG standard curve. No antibody production was observed for PBS or free ova-vaccinated mice, whereas the presence of anti-ova antibodies was detected for PPAA-ova vaccinated mice. This indicates that an antibody-mediated response is implicated in the tumor protection observed for these mice. Some degree of antibody response is expected, and has been observed in additional studies which also report enhanced CTL activation. The increased antibody response observed for the PPAA-ovalbumin compared to delivery of ovalbumin alone is likely a product of the enhanced stability and lengthened circulation time provided by the polymer. It may also be indicative of an enhanced CD4+ T-helper cell response, which occurs through the MHC2 antigen presentation pathway, since one of the primary functions of T-helper cells is to activate and support antibody production by B-cells.

In order to more closely investigate the role of enhanced CTL activation in the observed tumor protective immune response, MHC1/SIINFEKL tetramers were used to measure the number of ova-reactive CD8+T cells in the spleen by flow cytometry. See Example 4. MHC-1 tetramers are composed of 4 MHC-1 molecules bound to the class I peptide epitope, in this case the SIINFEKL peptide derived from ovalbumin, and conjugated to a fluorescent molecule. These tetramers therefore bind to the T-cell receptor of CD8+ T-cells that are reactive to ovalbumin. MHC-1 tetramers have been employed to evaluate CD8+ responses in vaccine strategies using ovalbumin, as well as other antigens.

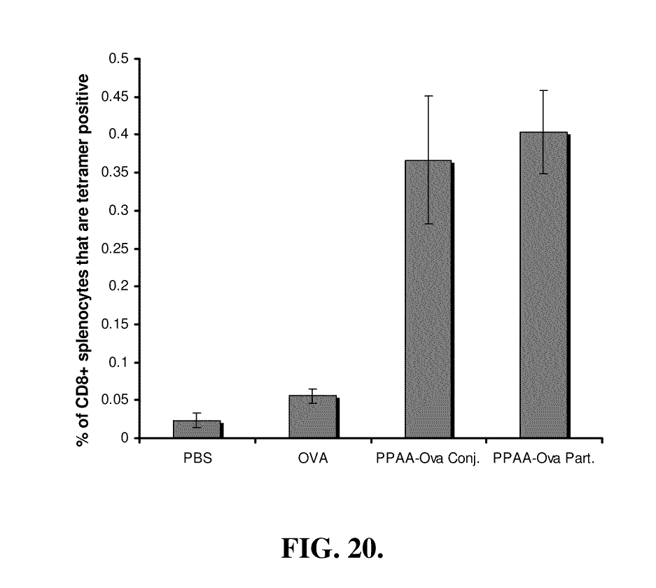

Mice (4 per group) were immunized with soluble and particulate PPAA-ovalbumin conjugates 7 days prior to sacrifice/splenocyte harvest. Mice immunized with either PBS, free ovalbumin, or PMAA soluble or particulate carriers were used as controls, and the experiment was performed 2 times independently. Delivery of free ovalbumin did not result in statistically significant CTL activation compared to PBS (p=0.31). Vaccination with soluble and particulate PPAA-ovalbumin conjugates resulted in enhanced CTL activation compared to PBS (p<0.0001), free ovalbumin (p<0.0002), and soluble PMAA-ovalbumin (p<0.0001), as evidenced by the increased number of CD8+/tetramer+ T-cells depicted in FIG. 20. Particulate PMAA-ovalbumin provided intermediate CTL activation. The CTL activation provided by the PMAA particle is likely due to the increased uptake and cross-presentation previously observed for particulate substances taken up by phagocytosis. CTL activation was slightly increased for particulate PPAA compared to soluble PPAA (p=0.29). An increase is expected based on the potential advantage of the particulate formulation to benefit from both increased uptake/presentation and cytosolic escape.

The ability of the PPAA protein antigen carrier to provide in vivo tumor protection was evaluated. It was concluded that both the soluble and particulate forms of the carrier provide strong protection against ovalbumin-expressing tumors. The anti-tumor immune response was found to consist of an increase in both anti-ova antibody production as well as anti-ova CTL activation. The PPAA-ova vaccines were able to prevent tumor growth for the duration of the 4-week study, with the exception of 1 mouse which had begun to develop at tumor on day 26, whereas control mice injected with PBS and free ovalbumin all developed tumors and had to be removed from the study by day 19.

Delivery of NY-ESO-1 Tumor Antigen to Primary Dendritic Cells Using Poly(Propylacrylic Acid) and Evaluation of Class I Presentation

In addition to the delivery of whole protein antigens in immunotherapeutic strategies, the specific MHC1 peptide epitope sequences can be delivered. This approach is advantageous in that the peptides are smaller in size and can be chemically synthesized, making them potentially easier to deliver in higher concentrations, as well as cheaper and more readily obtained than the full protein for most clinically relevant antigens. PPAA was used to deliver a peptide antigen to primary human dendritic cells (DCs). The peptide antigen to be delivered is the Class I HLA-A2 epitope (residues 157-165) of the NY-ESO-1 human tumor antigen, which is expressed by melanoma, ovarian, breast, and other cancers. The peptide was conjugated to the PPAA-PDSA polymer and delivered to primary human dendritic cells as described in Example 5. The resulting Class I antigen presentation was evaluated using an NY-ESO-1 (NY157-165)-specific CD8+ T cell clone. The PPAA carrier was able to induce Class I presentation of a peptide antigen in a dose dependent manner.

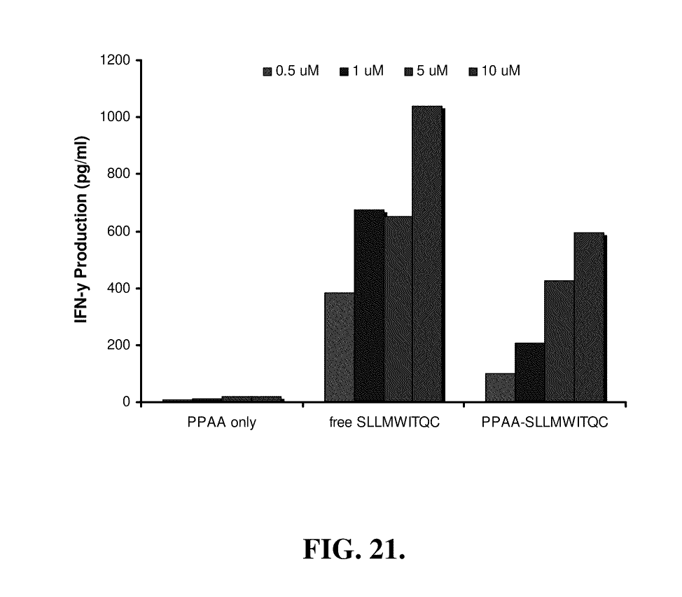

Polymer-peptide conjugation resulted in 80% of peptides reacting, according to the pyridine 2-thione measure. This corresponds to a conjugate composition of 14 wt % peptide and 86 wt % polymer. The evaluation shown in FIG. 21 compared PPAA-conjugated SLLMCWITQC (SEQ ID NO: 1) peptide with the free peptide, at increasing peptide concentrations. Conjugates or peptides were incubated with immature DCs, followed by DC maturation and incubation with HLA/peptide-specific CD8+ cells. The IFNγ produced by activated CD8+ cells was then measured by ELISA.

It can be seen that the conjugate does result in CTL activation in a dose-dependent manner, but not to the extent as that induced by the free peptide alone. However, since the free peptide can load externally into HLA molecules displayed on the DC surface, but the conjugate theoretically cannot due to steric hindrance, it is difficult to directly compare these two results. Another concern is that any unreacted peptide that was not purified away from the conjugate could bind the HLA externally and interfere with the results.

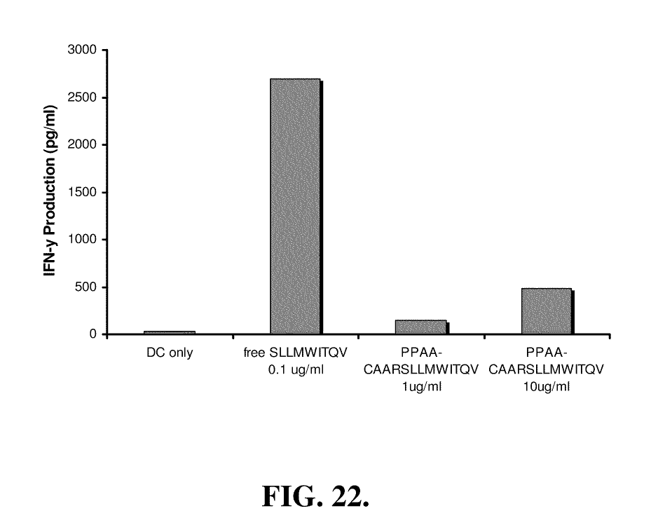

In order to address these issues, a second peptide sequence was employed: CAARSLLMWITQV (SEQ ID NO: 2). The natural peptide sequence, SLLMWITQC, was modified to give the alternate anchor residue valine. The sequence has been further modified to contain a cysteine residue, used for conjugation to the PDSA moiety of the PPAA polymer, attached to the peptide by a short linker sequence on the amine terminus end. The linker sequence culminates with an arginine residue, chosen based on studies reporting preferential cleaving after R residues by aminopeptidases in the ER and cytosol. This sequence therefore requires intracellular processing before it is able to bind the HLA molecule and induce CTL activation. It is also advantageous in that the polymer does not have to be conjugated to a residue that is part of the antigenic epitope. The results obtained with this conjugate are shown in FIG. 22, using the SLLMWITQV sequence as a positive control.

Some CTL activation is again seen, in a dose-dependent manner. The nature of the peptide sequence used indicates that the CTL activation seen is due to intracellular processing.

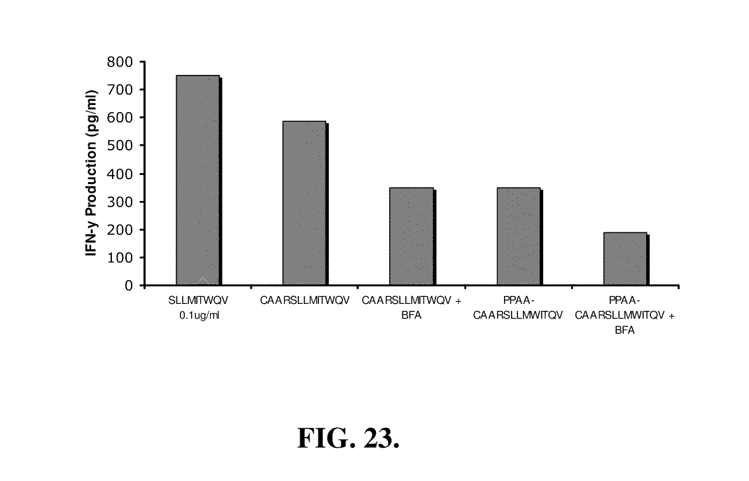

In a further attempt to distinguish between distinguish between activation due to antigens that have been internalized and processed vs. external peptide loading, the ER transport and vesicle transport inhibitor BFA was utilized. The results of this experiment are shown in FIG. 23. Some decrease in CTL activation was seen when the samples were treated with BFA, but a considerable degree of activation still occurred. Furthermore, reduction was observed to the same extent for the free peptide as for the conjugate, giving inconclusive results that suggest the method did not perform as expected. Also, the unconjugated CAARSLLMWITQV peptide gave activation levels higher than those of the conjugate.

The PPAA carrier was able to induce Class I presentation of a peptide antigen in a dose dependent manner. While external pulsing of DCs with free peptides is efficient for ex vivo strategies, in vivo delivery of injected peptide antigens will benefit from the stabilizing and concentrating effects provided by the carrier. The hydrophilic PPAA-PDSA polymer also confers solubility to the hydrophobic peptides, which normally are soluble only in organic solvents.

Protein immunotherapy is an important emerging strategy in the treatment of cancers. It offers significantly reduced toxicity compared to radiation and chemotherapy, is more tunable to specific cancers/patients, and provides a new means to combat cancers which show resistance to conventional treatments. Ongoing investigations in this area have revealed several opportunities for improving the efficacy of protein immunotherapies.

The present invention addresses the challenge of increasing protein antigen delivery to the cytosol of antigen presenting cells, where antigens are able to more efficiently induce a potent anti-tumor CTL response. Carriers based on the pH-sensitive, endosomally-active polymer poly(propylacrylic acid) were formulated to deliver the model protein antigen ovalbumin. The protein was conjugated through a disulfide linkage which is reversible by the cytosolic gluthathione reduction system, freeing the protein for the necessary processing. Both soluble and particulate architectures of the carrier are provided. An increase in MHC Class I presentation and subsequent CTL activation was demonstrated when both a macrophage cell line and a dendritic cell line was used as the antigen presenting cell. These increases in CTL activation were shown to correspond with the ability of the PPAA polymer to provide reduced exocytosis and thus increased intracellular accumulation over time.

These carriers also proved to provide excellent tumor protection in vivo, enhancing both the antibody and CTL-based immune responses. The in vivo advantages of the PPAA carrier are likely multi-faceted, as they not only cytosolic escape, but are also likely to provide serum stability and increased circulation time due to their hydrophilic and slightly negatively-charged state at physiological pH. They also promote increased uptake by APCs due to their larger size compared to delivery of free antigen. No carrier toxicity was observed either in vitro or in vivo, even at high concentrations, which is a key advantageous attribute. The carrier is also applicable to the delivery of peptide vaccines.

The present invention demonstrates the efficacy of PPAA-based carriers in the delivery of protein antigens for immunotherapeutic strategies. The carriers are highly suited to in vivo vaccine applications. Since the carrier has proven highly advantageous in the delivery of the model antigen ovalbumin, it can be expanded to the delivery of relevant human cancer antigens in mouse xenograft models. Furthermore, the success of the carriers in these evaluations indicates their applicability to a wide range of drug delivery processes which necessitate the delivery of protein or peptide therapeutics to targets in the cytosol of cells.

The following examples are provided for the purpose of illustrating, not limiting, the invention.

### EXAMPLES

### General Methods and Materials

All chemicals and reagents were ACS grade purchased from Sigma-Aldrich, St. Louis, Mo., and used without further purification unless otherwise noted.

Polymer compositions were determined by H¹—NMR using a Bruker AVance 300 MHz instrument and deuterated dimethyl sulfoxide (DMSO-d6, Fisher Chemical, Pittsburgh, Pa.). PDSA content was determined both by NMR and by the absorbance at 343 nm of pyridine-2-thione released from the polymer following reduction with excess of dithiothreitol (DTT). The molecular weight distribution was determined by GPC (Viscotek VE2001 sample module, VE3580 RI Detector, Waters Corp. ultrahydrogel columns) in 0.1 M sodium phosphate buffer, pH 8 using poly(ethylene oxide) (PEO) standards (Polysciences, Inc., Warrington, Pa.). The pKa of the PPAA-PDSA was determined by acid/base titration of 50 mg polymer in distilled, deionized water, starting at pH 12.5 and titrating with 0.1 N HCl.

Conjugates were characterized by GPC in 0.1M sodium phosphate buffer pH 8 using PEG standards. The final weight percent of ovalbumin in the conjugates was determined by the BCA protein assay (Pierce Biotechnology, Rockford, Ill.), using an ovalbumin standard curve. SDS-PAGE (polyacrylamide gel electrophoresis) was performed to detect any unreacted ovalbumin in the conjugate solution. Precast Tris-HCl polyacrylamide gels, 4-20% gradient, sample and running buffer, and protein standards were purchased from Bio-Rad, Hercules, Calif. Based on the results of the BCA assay, conjugates were loaded onto the gels in the appropriate amounts to give 10 ug of ovalbumin per lane. Gels were run for 45 min at 100 mA, then visualized using Coomassie staining (Bio-Rad, Hercules, Calif.).

### Example 1

### Preparation and Characterization of a Representative pH-Responsive Polymer Composition

### PPAA-Ovalbumin

PPAA-PDSA. For the synthesis of the PPAA-PDSA polymer, 0.007 mol propylacrylic acid (PAA) (Gateway Chemical Technology, St. Louis, Mo.), 0.00011 mol PDSA (synthesized according to previous protocols), and 0.000056 mol free-radical initiator azobisisobutyronitrile (AIBN, purified by recrystallization from methanol) were combined in a 5 ml flask and degassed by 4 rounds of freeze-vacuum-thaw then reacted at 60° C. for 24 hours. The polymer was dissolved in 3 ml dimethyl formamide (DMF) and purified by 3 rounds of precipitation in 500 ml diethyl ether.

PMAA-PDSA. Poly(methacrylic acid-co-PDSA) (PMAA-PDSA) was synthesized for use as a control polymer. It was formed by reversible addition fragmentation chain transfer polymerization (RAFT) by combining 0.016 mol methacrylic acid (MAA) (purified by distillation), 0.00022 mol PDSA, 0.000052 mol chain transfer agent 4-cyanopentanoic acid dithiobenzoate (CTP), 0.000052 mol initiator 2,2′-azobis(2,4-dimethyl valeronitrile) (V-65, Wako Chemicals USA, Richmond Va.) and 3 ml DMF in a 5 ml round bottom flask. The reaction was degassed by purging with N₂ for 20 min then polymerized at 40° C. for 24 hours. 6 ml DMF was added and the polymer was purified by 3 rounds of precipitation in 1 L diethyl ether.

Ovalbumin Conjugation. Conjugation was performed via disulfide exchange between the PDSA component of the polymer and free thiols introduced onto ovalbumin by reaction with Traut's reagent (2-iminothiolane, Pierce Biotechnology, Rockford, Ill.). 10 mg ovalbumin was mixed with a 10× molar excess of Traut's reagent in conjugation buffer (0.1M phosphate buffer, pH 7.8, 0.15M NaCl, 5 mM EDTA) for 1 hour at room temperature. The reaction mixture was purified using a PD-10 desalting column containing Sephadex G-25 (MWCO SkD, GE Healthcare, Piscataway, N.J.) and the degree of modification was estimated by Ellman's assay (Pierce Biotechnology, Rockford, Ill.). A 2.5× molar excess of polymer, either PPAA-PDSA or PMAA-PDSA, was immediately added to the modified protein and allowed to react 2 hours at room temperature in conjugation buffer. The degree of conjugation was estimated by measuring the absorbance at 343 nm (A₃₄₃) of the pyridine-2-thione group released from PDSA upon disulfide exchange, and the conjugate was purified on a PD-10 column and lyophilized for storage.

The membrane-disruptive PPAA-PDSA polymer was synthesized by free radical polymerization and resulted in a polymer with 3 mol % PDSA and M_{w}=26 kD, Mₙ=10 kD, and PDI=2.6, based on GPC analysis using PMMA standards. The pKa of this polymer was determined by acid/base titration to be 6.8, which is in the range of the transition between physiological and endosomal pH. A non membrane-disruptive polymer, PMAA-PDSA, of similar size (M_{w}34 kD, Mₙ=12 kD, PDI=2.8) containing 2 mol % PDSA was synthesized as a control. Methacrylic acid is less hydrophobic than propylacrylic acid in the protonated form and does not cause any membrane disruption in the RBC hemolysis assay.

The conjugation reaction can be controlled by adjusting the degree of protein thiolation and the duration of the conjugation reaction. A greater number of thiols on the protein, such as 5 or more, results in crosslinking and larger conjugates with complex structures. However, when the protein is modified to give an average of only 1-3 thiols per protein, conjugation does not occur as extensively. The degree of thiolation of the ovalbumin used to form the polymer-ovalbumin conjugates was determined by Ellman's assay to be an average of 3 per protein, or 15% of the total available lysines.

A BCA assay was performed to determine the final weight % of ovalbumin in each conjugate, using an ovalbumin standard curve, so that the samples could be normalized to contain the same amount of ovalbumin in the MHC-1 presentation assay. Both the PPAA-PDSA and PMAA-PDSA were found to contain 37 weight % ovalbumin and 63 weight % polymer, or 1.7 μg polymer per μg protein. GPC analysis gave M_{w}=200 kD, Mₙ=45 kD, PDI=4.4 for the PPAA-PDSA-ovalbumin conjugate and M_{w}=130 kD, Mₙ=57 kD, PDI=2.3 for the PMAA-PDSA-ovalbumin conjugate. SDS-PAGE was performed to ascertain that no free protein remained in the conjugate mixtures, as it could interfere with interpretation of the MHC-1 presentation assay results.

pH-Dependent Membrane-Disruptive Ability. The pH-dependent membrane-disruptive ability of the polymers and polymer-protein conjugates was estimated using a red blood cell hemolysis assay. Briefly, red blood cells were isolated and added to polymer and conjugate solutions (normalized to equivalent polymer amounts) of varying concentrations in 0.1M phosphate buffer at pH values of 5.8, 6.6, and 7.4. The degree of RBC membrane disruption (% hemolysis) was quantified by measuring the absorbance at 541 nm of the hemoglobin released into the solution by lysed cells, in comparison with complete lysis by Triton X-100 detergent. Sample concentrations were all normalized to contain 5 μg/ml of polymer, and samples were performed in triplicate with error reported as +/−one standard deviation.

MHC Class I Antigen Presentation Assay. The ability of the polymer to increase cytoplasmic delivery and subsequent MHC class I antigen presentation was evaluated using the lacZ antigen presentation assay. This assay utilizes a specialized LacZ B3Z CTL hybridoma. These CTLs produce β-galactosidase upon recognition of the ovalbumin class I antigenic epitope SIINFEKL complexed with the MHC class I molecule H-2K^{b}, present on RAW 309.1 CR macrophages. Therefore, a measure of β-galactosidase activity can be used to determine the degree to which delivered ovalbumin is presented as a class I antigen. All tissue culture reagents were purchased from Invitrogen Corp, Carlsbad, Calif., unless otherwise noted. RAW 309.1 CR macrophages (ATCC, Manassas, Va.) were cultured in 90% DMEM with D-Glucose and L-glutamine, 10% fetal bovine serum (FBS), supplemented with 100 U/ml penicillin/100 μg/ml streptomycin. B3Z CTLs were a gift from Dr. Nilabh Shastri, UC Berkeley. They were cultured in 90% RPMI medium with D-glucose and L-glutamine, 10% FBS, supplemented with 100 U/ml penicillin/100 μg/ml streptomycin, 50 μM 2-mercaptoethanol (Sigma-Aldrich, St. Louis, Mo.), and 1 mM sodium pyruvate. For the assay, RAW cells were plated at 5×10⁴ cells per well in a 96-well plate and grown overnight. PPAA-ovalbumin conjugates and control samples were added to the cells and incubated 6 hrs in DMEM with 5% FBS. Three sample concentrations were tested: 50 μg/ml, 100 μg/ml, and 150 μg/ml (samples were normalized to represent equivalent ovalbumin concentrations). RAW cells were rinsed with DMEM, then 1×10⁵ B3Z cells per well were added and incubated 16 hrs. Cells were rinsed with phosphate buffered saline then 100 μl lysis buffer [100 μM mercaptoethanol (Sigma-Aldrich, St. Louis, Mo.), 9 mM MgCl₂ (Sigma-Aldrich, St. Louis, Mo.), and 0.15 mM chlorophenol red β-D-galactoside (EMD Biosciences, San Diego, Calif.) in PBS] was added. After 4 hrs the absorbance of released chlorophenol red was measured at 595 nm. All samples were evaluated in triplicate and errors are reported as +/−one standard deviation. Statistical results (Student's t-test) are reported for the 100 μg/ml concentrations. The maximum possible β-galactosidase production was determined by chemically stimulating the B3Z cells in media containing 3.15 μM ionomycin (Sigma-Aldrich, St. Louis, Mo.) and 10 ng/ml phorbol 12-myristate 13-acetate (Sigma-Aldrich, St. Louis, Mo.) for 4 hours before rinsing and adding the lysis buffer.

Cytotoxicity Assay. The cytotoxicity of the PPAA and PMAA polymers and conjugates was determined for both the RAW and B3Z cell lines used in the MHC-1 presentation assay. Cytotoxicity was evaluated using the LDH (lactate dehydrogenase) assay (Roche Applied Sciences, Indianapolis, Ind.). This assay allows colorimetric measurement of LDH activity in the supernatant, which correlates to the proportion of dead or damaged cells. Cells were plated at 5×10⁴ cells/well in their normal culture media and polymer and conjugate samples were added to concentrations up to 300 μg/ml then incubated for 24 hrs. Cells were centrifuged at 250 g for 10 min, then 100 μl supernatant was removed and combined with 100 μl LDH reagent. The absorbance at 490 nm (reference 650 nm) was recorded every 5 min for 30 min and cell survival was determined by comparing to untreated cells and cells lysed with 1% Triton X-100 in water. Each sample was evaluated in triplicate and errors are reported as the standard error of the mean (SEM).

### Example 2

### Preparation and Characterization of ¹⁴C-Ovalbumin Conjugates

Preparation of PPAA/PMAA-¹⁴C-Ovalbumin Conjugates. Radioactively labeled ovalbumin-PPAA-PDSA and PMAA-PDSA conjugates were formed for tracking of the ovalbumin in cellular uptake and exocytosis experiments. The procedure used was similar to that described above for conjugate synthesis, except that a ¹⁴C label was added to ovalbumin. Ovalbumin was mixed with a 20× molar excess of Traut's reagent, followed by a 3× molar excess of ¹⁴C-iodoacetamide (MP Biomedical, Solon, Ohio). After reacting for 1 hour, a 2.5× molar excess of polymer, either PPAA or the negative control polymer PMAA, was added. Conjugates were again characterized by GPC in 0.1M sodium phosphate buffer pH 8 using PEG standards, and the final weight percent of ovalbumin was determined by a BCA assay. The amount of ¹⁴C per conjugate was determined by liquid scintillation counting, using a Beckman-Coulter LS600 liquid scintillation counter and EcoScint scintillation fluid (National Diagnostics, Atlanta, Ga.).

Cellular Uptake of PPAA-¹⁴C-Ovalbumin Conjugates. The cellular uptake and accumulation of ¹⁴C-labeled ovalbumin and the PPAA and PMAA-ovalbumin conjugates was studied in RAW 309.1 CR macrophages. Cells were plated in a 48-well plate at 75,000 cells/well and allowed to grow overnight. Either ovalbumin, PPAA-ovalbumin conjugate, PMAA-ovalbumin conjugate, or a PPAA and ovalbumin physical mixture was added to the cells at a concentration of 50 μg/ml of ovalbumin. Samples were incubated for either 15 min, 30 min, 1 hr, or 2 hrs. The cells were then washed 2× with PBS and lysed using 1% Triton X-100 in water. Radioactivity in the cell media, PBS wash, and cell lysate was measured using a Beckman-Coulter LS 6500 liquid scintillation counter. EcoScint scintillation fluid was obtained from National Diagnostics, Atlanta, Ga. Uptake of ¹⁴C-ovalbumin is presented as the % radioactivity present in the cell lysate compared to the total radioactivity delivered. The experiment was performed with a minimum of n=3.

Exocytosis Profiles of PPAA-¹⁴C-Ovalbumin Conjugates. The exocytosis of ¹⁴C-ovalbumin was measured using a procedure similar to that described by Besterman et al. RAW macrophages were plated at 75,000 cells/well in a 48-well plate and allowed to grow overnight. Either ovalbumin, PPAA-ovalbumin conjugate, PMAA-ovalbumin conjugate, or a PPAA and ovalbumin physical mixture was added to the cells at a concentration of 50 μg/ml of ovalbumin. Samples were incubated for either 1 min or 15 min, then uninternalized conjugate was removed and cells were washed 2× with media. Fresh media was added to the cells at the following timepoints: 5 min, 10 min, 20 min, 30 min, 1 hr, 2 hr, and 4 hr. The reappearance of ¹⁴C-ovalbumin into the supernatant was measured at each timepoint. After 4 hrs, cells were lysed with 1% Triton X-100 and the radioactivity in the lysate was measured. The amount of ovalbumin internalized after the 1 min or 15 min incubation time was determined as a percentage of the total delivered. The amount of ovalbumin exocytosed at each timepoint, as well as the amount remaining in the cells after 4 hrs, was then determined as a percentage of the total internalized. The experiments were performed with a minimum of n=3.

### Example 3

### Formulation and Characterization of Representative Ionic Particles

### PPAA-Ovalbumin/pDMAEMA

Ionic Particle Formation. Particles were formed by ionic complexation of the cationic pDMAEMA with the anionic PPAA-Ova conjugate. Conjugates were formed according to the procedures outlined in Examples 1 and 2. Briefly, ovalbumin was mixed with Traut's reagent, which reacts with lysine amines to give reactive SH groups. 10-15% of the 20 available lysines were modified. In the case of the radiolabeled conjugates used for the uptake/exocytosis studies, 30% of the amines were modified, to provide additional sites for attachment of the ¹⁴C label. The ovalbumin-SH was purified on a PD-10 desalting column, then reacted with the PDSA moiety on the PPAA or PMAA polymer. The conjugate was purified and exchanged to PBS using a Zeba desalting spin column, then stored at 4° C., omitting the lyophilization and freezing processes previously used. pDMAEMA was synthesized by RAFT (reversible addition fragmentation chain transfer) radical polymerization. DMAEMA monomer was dissolved in DMF at 33 wt. %. The chain transfer agent (CTA) 4-cyano-4-(ethylsulfanylthiocarbonyl)sulfanyl pentanoic acid (ECT) and initiator 2,2′-azobis(4-methoxy-2,4-dimethylvaleronitrile) (V-70) were added at a CTA:initiator ratio of 10:1. The solution was degassed with N² for 30 min, then reacted at 30° C. for 16 hrs and precipitated 3× in diethyl ether.

To form the particles, Ova-PPAA or PMAA conjugates were dissolved in PBS, pH 7.4. pDMAEMA was added at varying charge ratios calculated based on the % ionization and molar quantity of the pDMAEMA and PPAA or PMAA. Charge ratios ranging from neg:pos 40:1 to 1:3 were tested. The solution was vortexed then allowed to sit for 1 hour at room temperature for particle formation to occur. The particle size and zeta potential were determined in PBS using a Brookhaven (Holtsville, N.Y.) BI90Plus instrument equipped with a 535 channel correlator. Measurements were performed at a 90° angle with a 656 nm laser as the incident beam, and particle sizes are reported as the number average. Particle size standard deviations were calculated from the reported polydispersity according to the formula:

fwhm=Dₕ√μ₂

where Dₕ and μ₂ are the hydrodynamic diameter and polydispersity, respectively, reported by the particle sizer, and the fwhm (full-width at half maximum) is related to the standard deviation by: stdev=fwhm/[2√(21n2)].

In vitro Cytotoxicity of Ionic Particles. The cytotoxicity of PPAA-Ova and PMAA-Ova/pDMAEMA particles was tested in RAW macrophages using the Alamar Blue Cytoxocity Assay (Invitrogen, Carlsbad, Calif.). Alamar Blue allows quantification of cell viability by utilizing an oxidation-reduction indicator that fluoresces as growth medium undergoes chemical reduction resulting from cellular growth and metabolism. Particles were formed and characterized according to the procedure described in section 3.2.3. RAW macrophages were plated at 25,000 cells per well in 150 μl media (DMEM supplemented with 10% FBS) in a 96-well plate and incubated for 30 min at 37° C. 200 Alamar Blue reagent was added to each well. Then, Ova-PPAA and Ova-PMAA conjugates and particles were added to the cells at polymer concentrations of 100 μg/ml, 200 μg/ml, 500 μg/ml, and 750 μg/ml. Triton X-100 was used as a positive control for toxicity. Samples were incubated for 24 hrs, then fluorescence was measured using a Tecan Safire II plate reader using the following settings: Excitation: 545 nm w/bandwidth 6 nm, Emission: 590 nm w/bandwidth 20 nm, Gain: 68. % Survival compared to untreated cells was calculated for each sample. Samples were evaluated in triplicate and ANOVA statistical analysis was performed.

Cellular Uptake and Exocytosis of Ionic Particles. The uptake and exocytosis of particulate Ova-PPAA and PMAA conjugates into RAW macrophages was compared to their soluble counterparts according to the procedure described in sections 3.2.2 and 3.2.3. For uptake studies, cells were plated and grown overnight. The samples (ovalbumin, PPAA-ovalbumin conjugate, PMAA-ovalbumin conjugate, and PPAA-ovalbumin and PMAA-ovalbumin particles) were added to the cells at a concentration of 50 μg/ml of ovalbumin. Samples were incubated for 15 min, 30 min, 1 hr, or 2 hrs. The cells were then washed 2× with PBS and lysed, then radioactivity in the cell media, PBS wash, and cell lysate was measured. Uptake of ¹⁴C-ovalbumin is presented as the % radioactivity present in the cell lysate compared to the total radioactivity delivered. The experiment was performed with a minimum of n=3.

For exocytosis studies, cells were again plated and allowed to grow overnight. The samples (ovalbumin, PPAA-ovalbumin conjugate, PMAA-ovalbumin conjugate, and PPAA-ovalbumin and PMAA-ovalbumin particles) were added to the cells at a concentration of 50 μg/ml of ovalbumin. Samples were incubated for either 1 min or 15 min, then uninternalized conjugate was removed and cells were washed 2× with media. Fresh media was added to the cells at 5 min, 10 min, 20 min, 30 min, 1 hr, 2 hr, and 4 hr, and the reappearance of ¹⁴C-ovalbumin into the supernatant was measured. After 4 hrs, the cells were lysed and the radioactivity in the lysate was measured. The amount of ovalbumin internalized after the 1 min or 15 min incubation time was determined as a percentage of the total delivered. The amount of ovalbumin exocytosed at each timepoint, as well as the amount remaining in the cells after 4 hrs, was then determined as a percentage of the total internalized. The experiments were performed with a minimum of n=3.

MHC-1 Antigen Presentation and CTL Activation Assay in a Dendritic Cell Line. The ability of the particles to increase MHC class I antigen presentation in dendritic cells was evaluated using the lacZ CTL activation assay. The B3Z CTLs produce β-galactosidase upon recognition of the ovalbumin class I antigenic epitope SIINFEKL complexed with the MHC class 1 molecule H-2K^{b}, which is present on the DC2.4 cells. DC2.4 cells were cultured in 90% DMEM with D-Glucose and L-glutamine, 10% fetal bovine serum (FBS), supplemented with 100 μg/ml penicillin/100 μg/ml streptomycin. B3Z CTLs were a gift from Dr. Nilabh Shastri, UC Berkeley. They were cultured in 90% RPMI medium with D-Glucose and L-glutamine, 10% FBS, supplemented with 100 U/ml penicillin/100 μg/ml streptomycin, 50 μM 2-mercaptoethanol (Sigma-Aldrich, St. Louis, Mo.), and 1 mM sodium pyruvate. For the assay, DC2.4 cells were plated at 5×10⁴ cells per well in a 96-well plate and grown overnight. PPAA-ovalbumin conjugates and particles were added to the cells at an ovalbumin concentration of 100 μg/ml and incubated 6 hrs. Free ovalbumin was also tested at 100 μg/ml as well as a high concentration of 5 mg/ml. The cells were rinsed with DMEM, then 1×10⁵ B3Z cells per well were added and incubated 16 hrs. Cells were rinsed with PBS then 100 μl lysis buffer [100 μM mercaptoethanol (Sigma-Aldrich, St. Louis, Mo.), 9 mM MgCl₂ (Sigma-Aldrich, St. Louis, Mo.), and 0.15 mM chlorophenol red β-D-galactoside (EMD Biosciences, San Diego, Calif.) in PBS] was added to each well. After 2 hrs the absorbance of released chlorophenol red was measured at 595 nm. All samples were evaluated in triplicate and errors are reported as +/−one standard deviation.

### Example 4

### Representative pH-Responsive Polymer-Based Immunotherapeutic Agents for Tumor Protection

### PPAA-Ovalbumin

Sample Formulation. Soluble and particulate PPAA and PMAA ovalbumin conjugates were formulated to give a final concentration of 100 μg ovalbumin in a 150 μl injection volume. PPAA and PMAA conjugates and particles were freshly prepared according to the procedure described in Examples 2 and 3. A calculated −/+ charge ratio of 20:1 was used for particle formation. The size of the conjugates was determined by GPC and the size of the resulting pDMAEMA ionic particles was measured by DLS.

Tumor Protection Against Ovalbumin Expressing Tumors. The ability of PPAA-ovalbumin vaccines to prevent growth of ovalbumin expressing tumors was tested in mice. All studies were performed in compliance with the University of Washington Animal Care and Use Committee. Female C57B1/6 mice 7-8 weeks old were purchased from Jackson Labs (Bar Harbor, Me.). E.G7-OVA cells, which are derived from the same C57B1/6 inbred mouse strain, were obtained from ATCC (Manassas, Va.) and cultured in RPMI 1640 containing 10 mM Hepes, 1 mM sodium pyruvate, 4.5 g/L glucose, and 1.5 g/L sodium bicarbonate (ATCC, Manassas, Va.) supplemented with 0.05 mM 2-mercaptoethanol (Sigma), 0.4 mg/ml G418 (Invitrogen), and 10% FBS (Invitrogen). Mice were anesthetized with isofluorane and injected subcutaneously on the right flank using a 27 gauge needle. Mice (a minimum of 4 per group) were injected with 150 μl of PBS, ova, soluble PPAA-ova, particulate PPAA-ova, soluble PMAA-ova, or particulate PMAA-ova, with an equivalent of 100 μg ova delivered to each mouse. Seven days after vaccine administration, mice were again anesthetized and the hair on the left flank was removed using Nair. The E.G7-OVA tumor cells (1×10⁶ cells per mouse) were then injected in a volume of 1000 PBS subcutaneously into the left flank. Mice were monitored every 2-3 days and tumor length and width were measured using digital calipers (VWR, Brisbane, Calif.). The tumor volume was then calculated according to the formula:

Volume=0.5236×length×width

which is based on an ellipsoidal shape, with the height of the tumor estimated by the width, and has previously been used to approximate tumor volume. Mice were euthanized when tumor volume exceeded 2 cm³.

Determination of Antibody Response in Blood by ELISA. As part of the evaluation of the immune response to the administered vaccines, the IgG antibody response to ovalbumin was measured for all mice in the tumor study described in section 5.2.2. All reagents were obtained from Sigma Aldrich Corp. (St. Louis, Mo.) unless otherwise noted. Blood was collected by retro-orbital bleeding into heparin-coated capillary tubes (VWR, Brisbane Calif.). The blood was transferred to 0.5 ml eppendorf tubes and centrifuged for 10 min at 10,000 rpm. The plasma was then collected and diluted 1:5000 in PBS. An Enzyme-Linked ImmunoSorbent Assay (ELISA) was then performed to detect anti-ovalbumin IgG. First, 100 μl of a 5 μg/ml ovalbumin solution was added to each well of a Nunc Maxisorp 96-well plate and incubated overnight at 4° C. The plate was aspirated and blocked with 150 μl of a 1% bovine serum albumin (BSA) solution in PBS for 1.5 hrs at room temperature. The plate was then washed 1× and 100 μl of each diluted mouse sample was added. All samples were tested in triplicate. The samples were incubated for 3 hrs at room temperature, then the plate was washed 3×. Goat anti-mouse IgG (Fc Specific)-Peroxidase conjugate (Sigma product #A0168) was diluted 1:3000 in 0.1% BSA PBS-Tween and 100 μl was added to each well, followed by a 2 hr incubation at room temperature. The plate was washed 3× and 100 μl of SureBlue Reserve TMB peroxidase substrate (KPL Inc., Gaithersburg, Md.) was added. After 10 min, 100 μl 1N HCl was added and the absorbance at 450 nm was recorded. In order to generate a standard curve, mouse monoclonal anti-ovalbumin clone OVA-14 IgG1 (Sigma product #A6075) was used at concentrations of 3 ng/ml to 200 ng/ml. All washing steps were performed in PBS-Tween20 solution using a BioTek (Winooski, VT) ELx50 plate washer.

Determination of CD8+Response in Splenocytes Using MHC-1 Tetramers. In order to assess the ability of the PPAA carrier to provide CTL activation, MHC-1 tetramers were used to evaluate the splenocytes of immunized mice. Six-eight week old female C57B1/6 mice were obtained from Jackson Labs. Mice (4 per group) were anesthetized with isofluorane and immunized subcutaneously with 150 μl of PBS, ova, soluble PPAA-ova, particulate PPAA-ova, soluble PMAA-ova, or particulate PMAA-ova, with an equivalent of 100 μg ova delivered to each mouse. Seven days later mice were euthanized and their spleens harvested. Spleens were homogenized by forcing them through a 100 μm cell strainer into a Petri dish containing DMEM culture medium with L-glutamine and sodium pyruvate. Cells were counted and resuspended at 8×10⁶ cells/ml. Phycoerythrin (PE)-conjugated iTAg™ MHC class I tetramers were purchased from Immunomics/Beckman Coulter (Fullerton, Calif.), and the staining was carried out according to the provided protocol. 200 μl cell suspension was added to each flow cytometry tube. 5 μl Mouse BD FcBlock (BD Biosciences, San Jose, Calif.) was added and incubated for 5 min at 4C. Then, 10 μl of tetramer solution was added in addition to 10 μl FITC rat anti-mouse CD8a antibodies (BD Biosciences, San Jose, Calif.). Samples were vortexed gently and incubated for 30 min at room temperature. Red blood cells were then lysed by addition of 1 ml iTAg™ MHC Tetramer Lyse Reagent and 25 μl iTAg™ MHC Tetramer Fixative Reagent. Samples were vortexed 5 seconds and incubated for 10 min, then centrifuged at 150×g for 5 min. The supernatant was removed, 3 ml PBS was added, and the centrifugation step repeated. The cells were then resuspended in 500 μl PBS with 0.5% paraformaldehyde and stored at 4° C. for 1 hour before analysis on a FACScan 2 flow cytometer. Data was analyzed using FloJo software.

### Example 5

### Delivery of a Representative pH-Responsive Polymer Tumor Antigen Peptide to Primary Dendritic Cells and Evaluation of Class I Presentation

### PPAA-NY-ESO-1

Polymer-Peptide Conjugation. Peptides were obtained from EZBiolab (Westfield, Ind.). PPAA-PDSA was synthesized as described in the examples. Due to the hydrophobic nature of the peptide, the conjugation reaction was performed in organic solvent. 3 mg PPAA-PDSA (Mw=17 kD, Mn=6.2 kD, PDI=2.7) was dissolved in 150 μl dimethyl sulfoxide (DMSO). 0.53 mg of the Ny-Eso-1 peptide (MW 1.5 kD) peptide was then dissolved in 150 μl DMSO and added to the polymer solution. The reaction was carried out at room temperature overnight. The absorbance at 372 nm corresponding to released pyridine 2-thione was recorded to determine the extent of reaction. The conjugate was dialyzed into slightly basic water (MWCO 3.5 kD) for 1.5 days with frequent water changes, then lyophilized.

Evaluation of Class I Presentation in Human Primary Dendritic Cells. Peripheral blood mononuclear cells (PBMCs) were collected from HLA-A2+ donors. Dendritic cells (DCs) were generated by exposing adherent cells to 500 U/ml IL-4 and 800 U/ml GM-CSF in AIM-V culture medium (Invitrogen, Carlsbad, Calif.). 1×10⁵ DCs were then exposed to PPAA-peptide conjugate or free peptide for 4 hours. DCs were washed and matured by exposure to IL1β and LPS for 1 day, then incubated with 2×10⁵ KJ-4 NY157-specific CTL clones. After 18 hrs, the supernatant was collected and ELISA was performed to measure IFN-γ produced by activated CTLs.

Use of Brefeldin A to Investigate Intracellular Antigen Processing vs. External Peptide Loading. When DCs are pulsed with peptide, it is possible for the peptide to externally load empty MHC molecules displayed on the cell surface. In an attempt to distinguish between activation due to antigens that have been internalized and processed vs. external peptide loading, the ER transport and vesicle transport inhibitor Brefeldin A (BFA) was used. The experiment was performed according to the procedure described above with the addition of a group in which the DCs were treated with BFA prior to exposure to the peptide/conjugate.

While illustrative embodiments have been illustrated and described, it will be appreciated that various changes can be made therein without departing from the spirit and scope of the invention.

The embodiments of the invention in which an exclusive property or privilege is claimed are defined as follows:

1. A polymer conjugate, comprising:
(a) a pH-responsive polymer; and
(b) one or more immunotherapeutic agents covalently coupled thereto.

2. The conjugate of claim 1, wherein the pH-responsive polymer is a membrane destabilizing polymer or membrane disrupting polymer.

3. The conjugate of claim 1, wherein the pH-responsive polymer comprises plurality of repeating units comprising a C₂-C₈ alkyl group and a carboxylic acid group ionized at pH 7.4 and protonated at pH 5.5-6.0.

4. The conjugate of claim 1, wherein the pH-responsive polymer comprises a repeating unit having the formula: 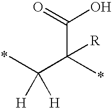
wherein * designates the point of attachment of the repeat unit to other repeat units and R is a C2-C8 alkyl group.

5. The conjugate of claim 1, wherein the pH-responsive polymer is a random, block, or graft copolymer.

6. The conjugate of claim 1, wherein the immunotherapeutic agent is a protein or peptide therapeutic agent.

7. The conjugate of claim 1, wherein the immunotherapeutic agent is a protein or peptide antigen.

8. The conjugate of claim 1, wherein the immunotherapeutic agent is a protein or peptide cancer antigen.

9. The conjugate of claim 1, wherein the immunotherapeutic agent is a protein or peptide vaccine.

10. A particle, comprising the conjugate of claim 1 and a cationic complexing agent.

11. A pharmaceutical composition, comprising a pharmaceutically acceptable excipient and a conjugate of claim 1.

12. A method for delivering a protein or peptide antigen to cell's cytosol, comprising contacting a cell with a conjugate of claim 1.

13. A method for inducing a cytotoxic T-lymphocyte response, comprising contacting a cell with the conjugate of claim 1.

14. A method for providing tumor protection to a subject, comprising administering to a subject a therapeutically effective amount of the conjugate of claim 1, wherein the immunotherapeutic agent is a protein or peptide cancer antigen.

15. A method for introducing a tumor-specific protein antigen to an antigen presenting cell to induce an immune response again the antigen and cells presenting the antigen, comprising contacting an antigen presenting cell with the conjugate of claim 1.

16. The method of claim 15, wherein the antigen presenting cells are selected from dendritic cells, macrophages, and B cells.
